(19) United States
(12) Patent Application Publication
(10) Pub. No.: US 2022/0235357 A1
(43) Pub. Date: Jul. 28, 2022

(54) COMPOSITIONS AND METHODS FOR IDENTIFYING AND INHIBITING A PAN-CANCER CELLULAR TRANSITION OF ADIPOSE-DERIVED STROMAL CELLS

(71) Applicants: The Trustees of Columbia University in the City of New York, New York, NY (US); The University of Texas Health Science Center at Houston, Houston, TX (US)

(72) Inventors: Dimitris Anastassiou, Englewood Cliffs, NJ (US); Lingyi Cai, New York, NY (US); Kaiyi Zhu, San Mateo, CA (US); Mikhail Kolonin, Houston, TX (US)

(73) Assignees: The Trustees of Columbia University in the City of New York, New York, NY (US); The University of Texas Health Science Center at Houston, Houston, TX (US)

(21) Appl. No.: 17/563,963

(22) Filed: Dec. 28, 2021

Related U.S. Application Data
(60) Provisional application No. 63/131,079, filed on Dec. 28, 2020.

(51) Int. Cl. C12N 15/113; C12Q 1/6886; G01N 33/574; C12N 15/11; C12N 9/22; A61P 35/00

(52) U.S. Cl. CPC C12N 15/1135; C12Q 1/6886; G01N 33/57484; C12N 15/11; C12Q 1/6869; A61P 35/00; C12Q 2600/158; C12Q 2600/156; C12N 9/22

(57) ABSTRACT

The present subject matter relates to the use of one or more inhibitors to treat a disease, e.g., cancer, in a subject. The presently disclosed subject matter provides for compositions and methods for treating a subject using a cancer transition inhibitor, an inhibitor that reduces the expression level of a marker of the transition of adipose-derived stromal cells (ASCs) to COL11A1-expressing cancer-associated fibroblasts (CAFs).

### CROSS-REFERENCE TO RELATED APPLICATIONS

This application claims priority to the U.S. Provisional Application Ser. No. 63/131,079, filed Dec. 28, 2020, which is hereby incorporated by reference in its entirety.

### INTRODUCTION

The present disclosure relates to the use of cancer transition inhibitor inhibitors that regulate the expression/activity level of a biomarker of the transition of adipose-derived stromal cells (ASCs) to COL11A1-expressing cancer-associated fibroblasts (CAFs) and pharmaceutical compositions thereof.

### SEQUENCE LISTING

The instant application contains a Sequence Listing which has been submitted electronically in ASCII format and is hereby incorporated by reference in its entirety. Said ASCII copy, created on Apr. 8, 2022, is named 070050_6578_SL.txt and is 2,943 bytes in size.

### BACKGROUND

Cancer-associated fibroblasts (CAFs) can be defined by their gene signature. For example, a cancer stage-associated COL11A1/INHBA/THBS2-expressing gene signature has been identified in CAFs present only after a particular staging threshold is reached. Specifically, COL11A1, INHBA, or THBS2 only appeared in ovarian cancer of at least stage III, in colon cancer of at least stage II, and in breast cancer of at least invasive stage I (but not in carcinoma in situ). Despite such efforts, however, there remains a need in the art for therapeutics targeting novel pathways and biomarkers for improved anti-cancer treatment that capitalize on our evolving understanding of how healthy cells transition into CAFs.

### SUMMARY

The disclosed subject matter relates to methods for treating cancer. The disclosed subject matter further provides pharmaceutical compositions for use according to the disclosed methods.

In certain embodiments, the disclosed subject matter provides methods for treating cancer in a subject. In certain embodiments, the method can include administering a therapeutically effective amount of a cancer transition inhibitor to the subject. In non-limiting embodiments, the cancer transition inhibitor can reduce the expression level of a marker of the transition of adipose-derived stromal cells (ASCs) to COL11A1-expressing cancer-associated fibroblasts (CAFs).

In certain embodiments, the cancer transition inhibitor is a polypeptide, a nucleic acid, a small molecule, or a combination of two or more thereof. In non-limiting embodiments, the cancer transition inhibitor can reduce the expression level of the marker by introducing an indel into the coding sequence of the marker. In non-limiting embodiments, the cancer transition inhibitor can be a transposase/transposon, a Zinc Finger nuclease, a TALEN, or an RNA-guided nuclease.

In certain embodiments, the marker can be a long non-coding ribonucleic acid (lncRNA), a micro ribonucleic acid (miRNA), RARRES1, SFRP4, COL11A1, INHBA, THBS2 or a combination thereof. In non-limiting embodiments, the lncRNA can be LINC01614, AC134312.5, AC009093.1, LINC01615, or combinations thereof. In non-limiting embodiments, the miRNA can be hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof.

The presently disclosed subject matter further provides pharmaceutical compositions for treating cancer. In certain embodiments, the pharmaceutical composition can include a therapeutically effective amount of a cancer transition inhibitor capable of reducing the expression level of a marker of a transition of ASCs to COL11A1-expressing CAFs and a pharmaceutically acceptable excipient. In non-limiting embodiments, the cancer transition inhibitor can be a polypeptide, a nucleic acid, a small molecule, or a combination of two or more thereof. In non-limiting embodiments, the marker can be an lncRNA, an miRNA, RARRES1, SFRP4, COL11A1, INHBA, THBS2 or a combination thereof. In non-limiting embodiments, the lncRNA can be LINC01614, AC134312.5, AC009093.1, LINC01615, or combinations thereof. In non-limiting embodiments, the miRNA can be hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof.

The presently disclosed subject matter further provides methods for determining the prognosis of a subject having cancer. In certain embodiments, the method can include obtaining a sample from the subject and determining an expression level of a marker related to a transition of ASCs to COL11A1-expressing CAFs, wherein increased expression of the marker in the sample relative to a reference control is indicative of a poor prognosis. In non-limiting embodiments, the marker can be an lncRNA, an miRNA, RARRES1, SFRP4, COL11A1, INHBA, THBS2 or a combination thereof. In non-limiting embodiments, the lncRNA can be LINC01614, AC134312.5, AC009093.1, LINC01615, or combinations thereof. In non-limiting embodiments, the miRNA can be hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof.

In certain embodiments, the sample can include tissue obtained from blood, bladder, breast, colon, brain, kidney, liver, lung, esophagus, gall-bladder, ovary, pancreas, stomach, cervix, thyroid, prostate, or skin of the subject.

In certain embodiments, the method can further include comparing the expression level of the marker of the sample with an expression level of a reference control, wherein the reference control is a healthy cell that has wild-type RARRES1, SFRP4, COL11A1, INHBA, and/or THBS2 expression.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIGS. 1A-1B. Scatter plots for fibroblast-rich samples for patients (A) T11 and (B) T23. Each dot represents a mesenchymal cell identified in the sample. The x- and y-axis denote the expression levels of COL11A1 and APOD, respectively. Dots are colored for the expression of fibroblastic marker LUM. The expression unit is the normalized log-transformed value from the count matrix (Materials and methods).

FIGS. 2A-2D. Trajectory analysis of pancreatic ductal adenocarcinoma (PDAC). (A) GAM fit to pseudotime ordered expression data to visualize the trend of gene expressions. (B) Expression of adipose-related genes along the transition lineage. The x-axis shows the cell orders, and the y axis shows the normalized read count. (C) Expression of COL11A1-associated genes along the transition lineage. (D) Expression of RARRES1 and SFRP4 genes along the transition lineage.

FIGS. 3A-3C. Overview of the PDAC fibroblasts. (A) 6,267 fibroblasts originated from 11 control pancreases, and 23 tumor samples were petitioned into four groups X1-X4. Fractions of the fibroblasts were: 45%, 38%, 14%, and 3%. (B) Table showing the top 20 DE genes for each cluster. (C) Bar plots presenting the numbers of cells captured for each cluster.

FIGS. 4A-4D. Trajectory analysis of 6,075 fibroblasts in the PDAC dataset. (A) Colors coded for pseudotime changing, red presenting the beginning of differentiation and blue presenting the end. (B) Color-coded trajectory analysis of fibroblasts for annotated three clusters. (C) Color-coded trajectory analysis of fibroblasts for group information. (D) Color-coded trajectory analysis of fibroblasts for sample identity.

FIGS. 5A-5D. Unsupervised clustering of four datasets from HNSCC, ovarian cancer, lung cancer and breast cancer. (A) t-SNE embedding of the whole HNSCC dataset. (B) t-SNE embedding of the whole ovarian cancer dataset. (C) t-SNE embedding of the mesenchymal cells from lung cancer dataset. (D) t-SNE embedding of the mesenchymal cells from breast cancer dataset.

FIG. 6. Immortalized ASC expressing tdTomato have higher LINC01614 and COL11A1 expression than primary adipose-derived stromal/stem cells (ASC). Gene mRNA expression of LINC01614 and COL11A1 measured by RT-PCR. Cancer cells express only the background level of LINC01614, COL11A1, and the marker of CAF.

FIG. 7. Immortalized ASC transduced for LINC01614 CRISPR KO and selected with Blasticidin and Puromycin (BP) have LINC01614 expression and dramatically reduced COL11A1 expression, as measured by RT-PCR.

FIG. 8. Change in LNC01614 KO ASC. Left: Image of imASC cultured with MDAMB231 cells. Right: Relative gene expression in imASC alone vs imASC cultured with MDAMB231 cells.

FIG. 9. Induction of LINC01614, SFRP4 and THBS2 expression in imASC co-cultured with SUM149 Top: Image of imASC cultured with SUM149 cells. Bottom: Relative gene expression in imASC alone vs imASC cultured with SUM149 cells.

### DETAILED DESCRIPTION

For clarity and not by way of limitation, the detailed description of the present disclosure is divided into the following subsections:
Definitions;
II. Biomarkers Related to the Transition of ASCs to COL11A1-Expressing CAFs and Cancer Transition Inhibitors;
III. Methods of Treatment; and
IV. Pharmaceutical Compositions.

### I. Definitions

The terms used in this specification generally have their ordinary meanings in the art, within the context of the present disclosure and in the specific context where each term is used. Certain terms are discussed below, or elsewhere in the specification, to provide additional guidance to the practitioner in describing the formulations and methods of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing,” and “comprising” are interchangeable, and one of the skills in the art is cognizant that these terms are open-ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, e.g., up to 10%, up to 5%, or up to 1% of a given value. Alternatively, particularly with respect to biological systems or processes, the term can mean within an order of magnitude, e.g., within 5-fold, or within 2-fold, of a value.

The term “agent,” as used herein, means a substance that produces or is capable of producing an effect and would include, but is not limited to, chemicals, pharmaceuticals, biologics, small organic molecules, antibodies, nucleic acids, peptides and proteins.

As used herein, the term “inhibitor” refers to a compound or molecule (e.g., small molecule, peptide, peptidomimetic, siRNA, antisense nucleic acid, aptamer, or antibody) that interferes with (e.g., reduces, prevents, or eliminates) the signaling function of a protein or pathway. An inhibitor can be any compound or molecule that changes any activity of a target or interferes with the interaction of a target with a signaling pathway. Inhibitors also include molecules that indirectly regulate the biological activity of a target by interacting with upstream signaling molecules.

An “anti-cancer effect” refers to one or more of: a reduction in aggregate cancer cell mass; a reduction in cancer cell growth rate; a reduction in cancer progression; a reduction in cancer cell proliferation. In certain embodiments, an anti-cancer effect can refer to a complete response, a partial response, a stable disease (without progression or relapse), a response with a later relapse, or progression-free survival in a patient diagnosed with cancer.

An “anti-cancer agent,” as used herein, can be any composition or component thereof that has an anti-cancer effect. Anti-cancer and anti-tumor agents include, but are not limited to, chemotherapeutic agents, radiotherapeutic agents, cytokines, anti-angiogenic agents, apoptosis-inducing agents, anti-cancer antibodies and/or agents which promote the activity of the immune system. In certain embodiments, anti-cancer agents can be radiotherapeutic agents. In certain embodiments, anti-cancer agents can be chemotherapeutic agents. Other non-limiting exemplary anti-cancer agents that can be used with the presently disclosed subject matter include tumor-antigen-based vaccines and chimeric antigen receptor T-cells.

“Antibody,” “fragment of an antibody,” or “antibody fragment” are used interchangeably to mean one or more fragments or portions of an antibody that retain the ability to specifically bind to a specific antigen (Holliger et al., Nat. Biotech. (2005) 23(9): 1126). The present antibodies may be antibodies and/or fragments thereof. Antibody fragments include Fab, F(ab′)2, scFv, disulfide-linked Fv, Fc, or variants and/or mixtures. The antibodies may be chimeric, humanized, single chain, or bi-specific. All antibody isotypes are encompassed by the present disclosure, including IgA, IgD, IgE, IgG, and IgM. Suitable IgG subtypes include IgG1, IgG2, IgG3 and IgG4. An antibody light or heavy chain variable region consists of a framework region interrupted by three hypervariable regions, referred to as complementarity determining regions (CDRs). The CDRs of the present antibodies or antigen-binding portions can be from a non-human or a human source. The framework of the present antibodies or antigen-binding portions can be human, humanized, non-human (e.g., a murine framework modified to decrease antigenicity in humans), or a synthetic framework (e.g., a consensus sequence).

As used herein, the term “contacting” a sample with a compound or molecule (e.g., one or more inhibitors, activators and/or inducers) refers to placing the compound in a location that will allow it to touch the sample, e.g., peripheral neurons. The contacting can be accomplished using any suitable methods. For example, contacting can be accomplished by adding the compound to a sample, e.g., contained with a tube or dish. Contacting can also be accomplished by adding the compound to a culture medium that includes the sample.

As used herein, the term “disease” refers to any condition or disorder that damages or interferes with the normal function of a cell, tissue, or organ.

The terms “detection” or “detecting” include any means of detecting, including direct and indirect detection.

An “effective amount” (or “therapeutically effective amount”) is an amount sufficient to affect a beneficial or desired clinical result upon treatment. In certain embodiments, a therapeutically effective amount refers to an amount that is able to achieve one or more of an anti-cancer effect, prolongation of survival and/or prolongation of the period until relapse. For example, and not by way of limitation, a therapeutically effective amount can be an amount of a compound (e.g., inhibitor) that minimizes, prevents, reduces and/or alleviates the symptoms of peripheral neuropathy, e.g., chemotherapy-induced peripheral neuropathy. A therapeutically effective amount can be administered to a subject in one or more doses. The therapeutically effective amount is generally determined by the physician on a case-by-case basis and is within the skill of one in the art. Several factors are typically taken into account when determining an appropriate dosage to achieve a therapeutically effective amount. These factors include age, sex and weight of the subject, the condition being treated, the severity of the condition and the form and effective concentration of the cells administered.

The terms “inhibiting,” “eliminating,” “decreasing,” “reducing,” or “preventing,” or any variation of these terms, referred to herein, include any measurable decrease or complete inhibition to achieve a desired result.

The term “in need thereof” would be a subject known or suspected of having or being at risk of developing a disease, e.g., cancer.

An “individual” or “subject” herein is a vertebrate, such as a human or non-human animal, for example, a mammal. Mammals include, but are not limited to, humans, primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

The terms “homology” or “homologous thereto,” as used herein, refer to the degree of homology between nucleic acid or amino acid sequences as determined using methods known in the art, for example, but not limited to, software such as BLAST or FASTA.

As used herein, a “protein” or “polypeptide” refers to a molecule that includes at least one amino acid residue.

As used herein, the term “treating” or “treatment” (and grammatical variations thereof such as “treat”) refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing metastases, decreasing the rate of disease progression, amelioration or palliation of the disease state and remission or improved prognosis. By preventing the progression of a disease or disorder, a treatment can prevent deterioration due to a disorder in an affected or diagnosed subject or a subject suspected of having the disorder, but also a treatment can prevent the onset of the disorder or a symptom of the disorder, e.g., peripheral neuropathy, in a subject at risk for the disorder or suspected of having the disorder. In certain embodiments, “treatment” can refer to a decrease in the severity of complications, symptoms, and/or cancer growth. For example, and not by way of limitation, the decrease can be a 10%, 20%, 30%, 40%, 50%, 60%, 70%, 80%, 90%, 95%, 98% or 99% decrease in the severity of complications, symptoms and/or cancer growth, for example, relative to a comparable control subject not receiving the treatment. In certain embodiments, “treatment” can also mean prolonging survival as compared to expected survival if treatment is not received.

As described herein, any concentration range, percentage range, ratio range, or integer range is to be understood to include the value of any integer within the recited range and, when appropriate, fractions thereof (such as one-tenth and one-hundredth of an integer), unless otherwise indicated.

### II. Biomarkers Related to a Transition of ASCs to COL11A1-Expressing CAFs and Cancer Transition Inhibitors

The present disclosure relates to the use of cancer transition inhibitors that regulate, directly or indirectly, the transition of adipose-derived stromal cells (ASCs) to COL11A1-expressing cancer-associated fibroblasts (CAFs) for preventing and/or treating a disease.

CAFs can be produced as a result of the interaction of tumor cells with the adipose microenvironment. For example, in certain embodiments, CAFs are produced by a pan-cancer cellular transition originating from ASCs naturally present in the stromal vascular fraction of normal adipose tissue. In certain embodiments, the ASCs can be characterized by a gene expression signature with the prominent presence of genes APOD, CXCL12, DPT, CFD, MGP, SEPRINE1, C1S, CCDC80, SRPX, COL1A2 COL6A3, or MMP2. In certain embodiments, CAFs can be characterized by a gene expression signature with the prominent presence of genes COL11A1, RARRES1, SFRP4, THBS2 and INHBA, or COL5A2. In addition, CAFs are associated with poor prognosis, invasiveness, metastasis and resistance to therapy in multiple cancer types.

The present disclosure identifies the continuous modification of the gene expression profiles of cells as they transition from ASCs (e.g., APOD-expressing adipose-derived stromal cells) to CAFs. In certain embodiments, the disclosed subject matter identifies specific biomarkers involved in the transition. For example, the biomarkers can include a long non-coding ribonucleic acid (lncRNA), a micro ribonucleic acid (miRNA), RARRES1, SFRP4, COL11A1, INHBA, THBS2 or combinations thereof.

Long Noncoding RNAs

In certain embodiments, the present disclosure relates to the use of a cancer transition inhibitor where the inhibitor inhibits one or more lncRNAs involved in the transition of ASCs to COL11A1-expressing CAFs. Exemplary lncRNAs include LINC01614, AC134312.5, AC009093.1, LINC01615, and combinations thereof. As used herein, the terms “LINC01614” and “LNC01614” are interchangeable.

Suitable inhibitors of lncRNAs known in the art can be used with the presently disclosed subject matter. Non-limiting exemplary inhibitors include any nucleic acids, proteins, and small molecules, as well as combinations thereof that reduce or eliminate the expression, function and/or activity of LINC01614, AC134312.5, AC009093.1, LINC01615, or combinations thereof. In certain embodiments, the inhibitor can reduce or eliminate the signaling pathway and/or activity of the lncRNAs. For example, but not by way of limitation, “inhibit” can refer to a reduction of at least 20%, at least 30%, at least 40%, at least 50%, at least 60%, at least 70%, at least 80%, at least 90%, at least 95%, at least 97% or at least 99% in the activity, function and/or expression of LINC01614, AC134312.5, AC009093.1, LINC01615, or combinations thereof, e.g., as compared to a reference. In certain embodiments, the reference is a sample from a healthy subject that is not treated with the lncRNA inhibitor.

In certain embodiments, the lncRNA inhibitor for use with the presently disclosed subject matter can be a small molecule compound that inhibits and/or eliminates the expression, function and/or activity of LINC01614, AC134312.5, AC009093.1, LINC01615, or combinations thereof or a pharmaceutically acceptable salt or solvate thereof.

In certain embodiments, the lncRNA inhibitor can be a ribozyme, an antisense oligonucleotide, an shRNA molecule, or an siRNA molecule that specifically inhibits or eliminates the expression, function, and/or activity of LINC01614, AC134312.5, AC009093.1, LINC01615, or combinations thereof.

In certain embodiments, the lncRNA inhibitor can be an antisense nucleic acid, an shRNA, or a siRNA that is complementary to at least a portion of a nucleic acid sequence of LINC01614, AC134312.5, AC009093.1, or LINC01615. The complementarity of the portion relative to the nucleic acid sequence of LINC01614, AC134312.5, AC009093.1, or LINC01615 can be at least about 50, at least about 55, at least about 60, at least about 65, at least about 70, at least about 75, at least about 80, at least about 85, at least about 90, at least about 95, or at least about 98 percent. The percent homology can be determined by, for example, BLAST or FASTA software. In certain embodiments, the complementary portion constitutes at least 10 nucleotides, at least 15 nucleotides, at least 20 nucleotides, at least 25 nucleotides, at least 30 nucleotides. In certain embodiments, the antisense nucleic acid, shRNA, or siRNA molecules have up to 15, up to 20, up to 25, up to 30, up to 35, up to 40, up to 45, up to 50, up to 75, or up to 100 nucleotides in length. In certain embodiments, the antisense nucleic acid, shRNA, or siRNA molecules comprise DNA or atypical or non-naturally occurring residues, for example, but not limited to, phosphorothioate residues.

In certain embodiments, the antisense nucleic acid, shRNA, or siRNA molecules disclosed herein can be expressed from a vector or produced chemically or synthetically. Methods for selecting an appropriate dsRNA or dsRNA-encoding vector are well known in the art for genes whose sequence is known (e.g., see Tuschl, T. et al. (1999); Elbashir, S. M. et al. (2001); Hannon, G J. (2002); McManus, M T. et al. (2002); Brummelkamp, T R. et al. (2002); U.S. Pat. Nos. 6,573,099 and 6,506,559; and PCT Patent Application Nos. WO 2001/036646, WO 1999/032619 and WO 2001/068836, the contents of which are incorporated by reference herein in their entireties).

In certain embodiments, the lncRNA inhibitor for use with the presently disclosed subject matter can be a peptide and/or a protein fragment. For example, but not by way of limitation, a peptide and/or a protein fragment that inhibits or eliminates the expression, function and/or activity of LINC01614, AC134312.5, AC009093.1, LINC01615, or combinations thereof and/or partially or completely blocks the signaling pathway or activity of LINC01614, AC134312.5, AC009093.1, LINC01615, or combinations thereof.

In certain embodiments, the lncRNA inhibitor for use with the presently disclosed subject matter can be a transposase/transposon, a Zinc Finger nuclease, a TALEN, or an RNA-guided nuclease that can inhibit and/or reduce the expression, function and/or activity of LINC01614, AC134312.5, AC009093.1, LINC01615, or combinations thereof and/or partially or completely blocks the signaling pathway or activity of LINC01614, AC134312.5, AC009093.1, LINC01615, or combinations thereof. In non-limiting embodiments, the expression level of the marker can be reduced by introducing an indel into the coding sequence of the marker.

In certain embodiments, the lncRNA inhibitor can be an antibody or an antibody fragment that partially or completely blocks signaling and/or activity of LINC01614, AC134312.5, AC009093.1, LINC01615, or combinations thereof. For example, but not by way of limitation, an antibody (or fragment thereof) for use in the present disclosure can physically bind to LINC01614, AC134312.5, AC009093.1, or LINC01615 and/or bind to a protein that regulates the expression, activity and/or function of LINC01614, AC134312.5, AC009093.1, or LINC01615.

In certain embodiments, a lncRNA inhibitor of the present disclosure can be conjugated to a modality that specifically targets cancer cells. For example, and not by way of limitation, the lncRNA inhibitor can be conjugated to an antibody or antibody fragment and/or peptide, e.g., that recognizes an epitope on the surface of a cancer cell. In certain embodiments, the modality can be a nanoparticle that specifically targets cancer cells, e.g., by the presence of a targeting moiety conjugated to the nanoparticle.

MicroRNAs

The present disclosure relates to the use of a cancer transition inhibitor of miRNAs involved in the transition of ASCs to COL11A1-expressing CAFs. Exemplary miRNAs include hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof.

Any suitable inhibitors of the miRNAs known in the art can be used with the presently disclosed subject matter. Non-limiting exemplary inhibitors can include any compounds, nucleic acids, proteins, and small molecules, as well as combinations thereof, that inhibit or eliminate the expression, function and/or activity of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof. In certain embodiments, the inhibitor can inhibit or eliminate inhibit the signaling pathway and/or activity of the miRNAs. For example, but not by way of limitation, “inhibit” can refer to a reduction of at least 20%, at least 30%, at least 40%, at least 50%, at least 60%, at least 70%, at least 80%, at least 90%, at least 95%, at least 97% or at least 99% in the activity, function and/or expression of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof, e.g., as compared to a reference. In certain embodiments, the reference is a sample from a healthy subject not treated with the miRNA inhibitor.

In certain embodiments, the miRNA inhibitor for use with the presently disclosed subject matter can be a small molecule compound that inhibits or eliminates the expression, function and/or activity of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof or a pharmaceutically acceptable salt or solvate thereof.

In certain embodiments, the miRNA inhibitor can be a ribozyme, an antisense oligonucleotide, an shRNA molecule, and/or a siRNA molecule that specifically inhibits or eliminates the expression, function and/or activity of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof.

In certain embodiments, the miRNA inhibitor can be an antisense nucleic acid, an shRNA, or a siRNA that is complementary to at least a portion of a nucleic acid sequence of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof. The complementarity of the portion relative to the nucleic acid sequence of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof can be at least about 50, at least about 55, at least about 60, at least about 65, at least about 70, at least about 75, at least about 80, at least about 85, at least about 90, at least about 95, or at least about 98 percent. The percent complementarity can be determined by, for example, BLAST or FASTA software. In certain embodiments, the complementary portion constitutes at least 10 nucleotides, at least 15 nucleotides, at least 20 nucleotides, at least 25 nucleotides, at least 30 nucleotides. In certain embodiments, the antisense nucleic acid, shRNA, or siRNA molecules have up to 15, up to 20, up to 25, up to 30, up to 35, up to 40, up to 45, up to 50, up to 75, or up to 100 nucleotides in length. In certain embodiments, the antisense nucleic acid, shRNA, or siRNA molecules comprise DNA or atypical or non-naturally occurring residues, for example, but not limited to, phosphorothioate residues. In certain embodiments, the antisense nucleic acid, shRNA, or siRNA molecules disclosed herein can be expressed from a vector or produced chemically or synthetically.

In certain embodiments, the miRNA inhibitor for use with the presently disclosed subject matter can be a peptide or a protein fragment. For example, but not by way of limitation, a peptide and/or a small protein fragment that inhibits or eliminates the expression, function and/or activity of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof, and/or partially or completely blocks the signaling pathway or activity of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof can be used as the inhibitor.

In certain embodiments, the miRNA inhibitor for use with the presently disclosed subject matter can be a transposase/transposon, a Zinc Finger nuclease, a TALEN, or an RNA-guided nuclease that can inhibit or eliminates the expression, function and/or activity of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof, or combinations thereof and/or partially or completely blocks the signaling pathway or activity of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof. In non-limiting embodiments, the expression level of the marker can be reduced by introducing an indel into the coding sequence of the marker.

In certain embodiments, the miRNA inhibitor can be an antibody or an antibody fragment that partially or completely blocks signaling and/or activity of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof. For example, but not by way of limitation, an antibody (or fragment thereof) for use in the present disclosure can physically bind to hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, or hsa-mir-214, and/or bind to a protein that regulates the expression, activity and/or function of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214.

In certain embodiments, a miRNA inhibitor of the present disclosure can be conjugated to a modality that specifically targets cancer cells. For example, and not by way of limitation, the miRNA inhibitor can be conjugated to an antibody or antibody fragment and/or peptide, e.g., that recognizes an epitope on the surface of a cancer cell. In certain embodiments, the modality can be a nanoparticle that specifically targets cancer cells, e.g., by the presence of a targeting moiety conjugated to the nanoparticle.

Additional Targets

The present disclosure relates to the use of a cancer transition inhibitor of additional targets involved in the transition of ASCs to COL11A1-expressing CAFs. Example additional targets include RARRES1, SFRP4, COL11A1, INHBA, THBS2, or combinations thereof.

Suitable inhibitors of the additional targets known in the art can be used with the presently disclosed subject matter. Non-limiting exemplary inhibitors can include any nucleic acids, proteins, and small molecules or combinations thereof that inhibit or eliminate the expression, function and/or activity of RARRES1, SFRP4, COL11A1, INHBA, THBS2, or combinations thereof. In certain embodiments, the inhibitor can inhibit or eliminate the signaling pathway and/or activity of the additional targets. For example, but not by way of limitation, “inhibit” can refer to a reduction of at least 20%, at least 30%, at least 40%, at least 50%, at least 60%, at least 70%, at least 80%, at least 90%, at least 95%, at least 97% or at least 99% in the activity, function and/or expression of RARRES1, SFRP4, COL11A1, INHBA, THBS2, or combinations thereof, e.g., as compared to a reference. In certain embodiments, the reference is a sample from a healthy patient not treated with the additional target inhibitor.

In certain embodiments, the additional target inhibitor for use with the presently disclosed subject matter can be a small molecule compound that inhibits or eliminates the expression, function and/or activity of RARRES1, SFRP4, COL11A1, INHBA, THBS2, or combinations thereof or a pharmaceutically acceptable salt or solvate thereof.

In certain embodiments, the additional target inhibitor can be a ribozyme, an antisense oligonucleotide, an shRNA molecule, and/or a siRNA molecule that specifically inhibits or eliminates the expression, function and/or activity of RARRES1, SFRP4, COL11A1, INHBA, THBS2, or combinations thereof.

In certain embodiments, the additional targets inhibitor can be an antisense nucleic acid, an shRNA, or a siRNA that is complementary to at least a portion of a nucleic acid sequence of RARRES1, SFRP4, COL11A1, INHBA, THBS2, or combinations thereof. The complementarity of the portion relative to the nucleic acid sequence of RARRES1, SFRP4, COL11A1, INHBA, THBS2, or combinations thereof can be at least about 50, at least about 55, at least about 60, at least about 65, at least about 70, at least about 75, at least about 80, at least about 85, at least about 90, at least about 95, or at least about 98 percent. The percent complementarity can be determined by, for example, BLAST or FASTA software. In certain embodiments, the complementary portion constitutes at least 10 nucleotides, at least 15 nucleotides, at least 20 nucleotides, at least 25 nucleotides, at least 30 nucleotides. In certain embodiments, the antisense nucleic acid, shRNA, or siRNA molecules have up to 15, up to 20, up to 25, up to 30, up to 35, up to 40, up to 45, up to 50, up to 75, or up to 100 nucleotides in length. In certain embodiments, the antisense nucleic acid, shRNA, or siRNA molecules comprise DNA or atypical or non-naturally occurring residues, for example, but not limited to, phosphorothioate residues. In certain embodiments, the antisense nucleic acid, shRNA, or siRNA molecules disclosed herein can be expressed from a vector or produced chemically or synthetically.

In certain embodiments, the additional target inhibitor for use with the presently disclosed subject matter can be a peptide and/or a protein fragment. For example, but not by way of limitation, a peptide and/or a protein fragment that inhibits or eliminates the expression, function and/or activity of RARRES1, SFRP4, COL11A1, INHBA, THBS2, or combinations thereof, and/or partially or completely blocks the signaling pathway or activity of RARRES1, SFRP4, COL11A1, INHBA, THBS2, or combinations thereof can be used as the inhibitor.

In certain embodiments, the additional target inhibitor for use with the presently disclosed subject matter can be a transposase/transposon, a Zinc Finger nuclease, a TALEN, or an RNA-guided nuclease that can inhibit or eliminates the expression, function and/or activity of RARRES1, SFRP4, COL11A1, INHBA, THBS2, or combinations thereof, or combinations thereof and/or partially or completely blocks the signaling pathway or activity of RARRES1, SFRP4, COL11A1, INHBA, THBS2, or combinations thereof. In non-limiting embodiments, the expression level of the marker can be reduced by introducing an indel into the coding sequence of the marker.

In certain embodiments, the additional target inhibitor can be an antibody or an antibody fragment that partially or completely blocks signaling and/or activity of RARRES1, SFRP4, COL11A1, INHBA, THBS2, or combinations thereof. For example, but not by way of limitation, an antibody (or fragment thereof) for use in the present disclosure can physically bind to RARRES1, SFRP4, COL11A1, INHBA, or THBS2, and/or bind to a protein that regulates the expression, activity and/or function of RARRES1, SFRP4, COL11A1, INHBA, THBS2, or combinations thereof.

In certain embodiments, the additional target inhibitors of the present disclosure can be conjugated to a modality that specifically targets cancer cells. For example, and not by way of limitation, the additional target inhibitor can be conjugated to an antibody or antibody fragment and/or peptide, e.g., that recognizes an epitope on the surface of a cancer cell. In certain embodiments, the modality can be a nanoparticle that specifically targets cancer cells, e.g., by the presence of a targeting moiety conjugated to the nanoparticle.

### III. Methods of Prognosis & Treatment

Prognostic Methods

The present disclosure provides methods for determining the prognosis of a patient that has cancer. As described in detail in the Example section below, the studies presented in the instant application indicate that high expression of the disclosed lncRNAs, miRNAs, and additional targets are associated with a poor prognosis.

In certain embodiments, the expression level of LINC01614, AC134312.5, AC009093.1, LINC01615, or combinations thereof, can be used as a marker for determining the prognosis of a subject. In certain embodiments, the expression level of hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof can be used as a marker for determining the prognosis of a subject. In certain embodiments, the expression level of RARRES1, SFRP4, COL11A1, INHBA, THBS2 or a combination thereof can be used as a marker for determining the prognosis of a subject.

In certain embodiments, a prognostic method of the present disclosure includes determining the expression level of the disclosed lncRNAs, miRNAs, or additional targets in a sample of a subject. In non-limiting embodiments, the sample can include any tissues or cells obtained from blood, bladder, breast, colon, brain, kidney, liver, lung, esophagus, gall-bladder, ovary, pancreas, stomach, cervix, thyroid, prostate, skin, or any other tissues of the subject. In certain embodiments, the method can further include comparing the expression level of the disclosed lncRNAs, miRNAs, or additional targets in the sample to a reference control level of the disclosed lncRNAs, miRNAs, or additional targets, where increased expression of the disclosed lncRNAs, miRNAs, or additional targets in the sample compared to the reference control level can indicate that the subject has a poor prognosis. A “reference control level” or “reference control expression level” of the disclosed lncRNAs, miRNAs, or additional targets, as used interchangeably herein, can, for example, be established using a reference control sample. Non-limiting examples of reference control samples include normal and/or healthy cells, e.g., from a sample of normal or benign tissue, that have a wild-type activity level of the disclosed lncRNAs, miRNAs, or additional targets. In certain embodiments, a reference control level can, for example, be established using normal cells, e.g., benign cells located adjacent to the tumor in a patient. In certain embodiments, the expression level of the disclosed lncRNAs, miRNAs, or additional targets can be the nucleic acid expression level. Alternatively and/or additionally, the expression level of the disclosed lncRNAs, miRNAs, or additional targets can be the protein expression level of the disclosed lncRNAs, miRNAs, or additional targets.

Where comparisons to reference control expression levels are referred to herein, the expression level of nucleic acid and/or protein of interest is assessed relative to the reference control expression level within the same species. For example, an expression level and/or presence of the disclosed human lncRNAs, miRNAs, or additional targets are compared with a human healthy reference control level.

In certain embodiments, the absence and/or a reduced expression of the disclosed human lncRNAs, miRNAs, or additional targets means the detection of less than about 90%, less than about 80%, less than about 70%, less than about 60%, less than about 50/%, less than about 40% or less than about 30% expression relative to the reference control level. In certain embodiments, the increased expression of the disclosed human lncRNAs, miRNAs, or additional targets means the detection of greater than about a 1.1-fold increase, greater than about a 1.2-fold increase greater than about a 1.5-fold increase greater than about a 1.75-fold increase greater than about a 2-fold increase greater than about a 2-fold increase, greater than about a 2.5-fold increase, greater than about 3-fold increase or greater than about a 3.5-fold increase in expression relative to the reference control level.

Methods for qualitatively and quantitatively detecting and/or determining the expression level of a nucleic acid can include, but are not limited to a polymerase chain reaction (PCR), including conventional, qPCR and digital PCR, in situ hybridization (for example, but not limited to Fluorescent In situ Hybridization (“FISH”)), gel electrophoresis, sequencing and sequence analysis, microarray analysis and other techniques known in the art. In non-limiting embodiments, lncRNAs can be detected through microarrays or RNA sequencing (RNA-seq) using next-generation sequencers. In non-limiting embodiments, miRNAs can be detected through Northern blot, RT-qPCR, sequencing, or biochip.

In certain embodiments, the method of detection can be real-time PCR (RT-PCR), quantitative PCR, fluorescent PCR, RT-MSP (RT methylation-specific polymerase chain reaction), PicoGreen™ (Molecular Probes, Eugene, Oreg.) detection of DNA, radioimmunoassay or direct radio-labeling of DNA. For example, but not by way of limitation, a nucleic acid can be reversed transcribed into cDNA followed by polymerase chain reaction (RT-PCR); or, a single enzyme can be used for both steps as described in U.S. Pat. No. 5,322,770, or the nucleic acid can be reversed transcribed into cDNA followed by symmetric gap ligase chain reaction (RT-AGLCR) as described by R. L. Marshall, et al., PCR Methods and Applications 4: 80-84 (1994).

In certain embodiments, quantitative real-time polymerase chain reaction (qRT-PCR) is used to evaluate mRNA levels. The levels of an mRNA of interest and a control mRNA can be quantitated in cancer tissue or cells and adjacent benign tissues.

In certain embodiments, the method of detection of the present disclosure can be carried out without relying on amplification, e.g., without generating any copy or duplication of a target sequence, without the involvement of any polymerase, or without the need for any thermal cycling. In certain embodiments, detection can be performed using the principles set forth in the QuantiGene™ method described in U.S. application Ser. No. 11/471,025, filed Jun. 19, 2006, and is incorporated herein by reference.

In certain embodiments, in situ hybridization visualization can be employed, where a radioactively labeled anti sense RNA probe is hybridized with a thin section of a biological sample, e.g., a biopsy sample washed, cleaved with Rnase, and exposed to a sensitive emulsion for autoradiography. The samples can be stained with hematoxylin to demonstrate the histological composition of the sample, and dark field imaging with a suitable light filter shows the developed emulsion. Non-radioactive labels such as digoxigenin can also be used.

In certain non-limiting embodiments, evaluation of nucleic acid expression can be performed by fluorescent in situ hybridization (FISH). FISH is a technique that can directly identify a specific region of DNA or RNA in a cell and therefore enables visual determination of the biomarker expression in tissue samples. The FISH method has the advantages of a more objective scoring system and the presence of a built-in internal control consisting of the biomarker gene signals present in all non-neoplastic cells in the same sample. FISH is a direct in situ technique that can be relatively rapid and sensitive and can also be automated. Immunohistochemistry can be combined with a FISH method when the expression level of the biomarker is difficult to determine by FISH alone.

In certain embodiments, the expression of a nucleic acid can be detected on a DNA array, chip or a microarray. Oligonucleotides corresponding to the nucleic acids of interest are immobilized on a chip which is then hybridized with labeled nucleic acids of a biological sample, e.g., a tumor sample, obtained from a subject. A positive hybridization signal is obtained with the sample containing the nucleic acids of interest. Methods of preparing DNA arrays and their use are well known in the art. (See, for example, U.S. Pat. Nos. 6,618,6796; 6,379,897; 6,664,377; 6,451,536; 548,257; U.S. Patent Application Nos. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See, for example, U.S. Patent Application No. 20030215858).

In certain embodiments, to monitor a target nucleic acid expression levels, a nucleic acid can be extracted from the biological sample to be tested, reverse transcribed, and fluorescent-labeled cDNA probes can be generated. The labeled cDNA probes can then be applied to microarrays capable of hybridizing to a biomarker, allowing hybridization of the probe to microarray and scanning the slides to measure fluorescence intensity. This intensity correlates with the hybridization intensity and expression levels of the biomarker.

Types of probes for the detection of nucleic acids include cDNA, riboprobes, synthetic oligonucleotides and genomic probes. The type of probe used will generally be dictated by the particular situation, such as riboprobes for in situ hybridization and cDNA for Northern blotting, for example. In certain non-limiting embodiments, the probe is directed to nucleotide regions unique to the particular RNA. The probes can be as short as is required to differentially recognize the particular biomarker mRNA transcripts and can be as short as, for example, 15 bases. Probes of at least 17 bases, 18 bases and 20 bases can also be used. In certain embodiments, the primers and probes hybridize specifically under stringent conditions to a nucleic acid fragment having the nucleotide sequence corresponding to the target gene. As herein used, the term “stringent conditions” means hybridization will occur only if there is at least 95% or at least 97% identity between the sequences.

The form of labeling of the probes can be any that is appropriate, such as the use of radioisotopes, for example, ³²P and ³⁵S, or fluorophores. Labeling with radioisotopes can be achieved, whether the probe is synthesized chemically or biologically, by the use of suitably labeled bases.

Methods for detecting and/or determining the expression level of a protein are well known to those skilled in the art, and include, but are not limited to, mass spectrometry techniques, I-D or 2-D gel-based analysis systems, chromatography, enzyme-linked immunosorbent assays (ELISAs), radioimmunoassays (MA), enzyme immunoassays (EIA), Western Blotting, immunoprecipitation and immunohistochemistry. These methods use antibodies, or antibody equivalents, to detect protein or use biophysical techniques. Antibody arrays or protein chips can also be employed, see, for example, U.S. Patent Application Nos. 2003/0013208; 2002/0155493, 2003/0017515 and U.S. Pat. Nos. 6,329,209 and 6,365,418, herein incorporated by reference in their entireties.

In certain non-limiting embodiments, a detection method for measuring protein expression the target biomarkers includes the steps of contacting a biological sample, e.g., a tissue sample, with an antibody or variant (e.g., fragment) thereof, which selectively binds the biomarker, and detecting whether the antibody or variant thereof is bound to the sample. The method can further include contacting the sample with a second antibody, e.g., a labeled antibody. The method can further include one or more washing steps, e.g., to remove one or more reagents. For example, but not by way of limitation, an antibody or variant (e.g., fragment) thereof for use in the methods disclosed herein can specifically bind to the disclosed target biomarkers. In certain embodiments, an antibody or variant (e.g., fragment) thereof for use in the methods disclosed herein can specifically bind to the target biomarkers.

In certain non-limiting embodiments, Western blotting can be used for detecting and quantitating protein expression levels. Cells can be homogenized in lysis buffer to form a lysate and then subjected to SDS-PAGE and blotting to a membrane, such as a nitrocellulose filter. Antibodies (unlabeled) can then be brought into contact with the membrane and assayed by a secondary immunological reagent, such as labeled protein A or anti-immunoglobulin (suitable labels including ¹²⁵I, horseradish peroxidase and alkaline phosphatase). Chromatographic detection can also be used. In certain embodiments, immunodetection can be performed with an antibody to a biomarker using the enhanced chemiluminescence system (e.g., from PerkinElmer Life Sciences, Boston, Mass.).

Immunohistochemistry can be used to detect the expression and/or presence of a biomarker, e.g., in a biopsy sample. A suitable antibody can be brought into contact with, for example, a thin layer of cells, followed by washing to remove unbound antibody, and then contacted with a second, labeled antibody. Labeling can be by fluorescent markers, enzymes, such as peroxidase, avidin or radiolabeling. The assay can be scored visually, using microscopy, and the results can be quantitated. Machine-based or auto imaging systems can also be used to measure immunostaining results for the biomarker.

Various automated sample processing, scanning and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples. See, e.g., the CAS-200 system (Becton, Dickinson & Co.).

Labeled antibodies against proteins can also be used for imaging purposes, for example, to detect the presence of the protein in cells of a subject. Suitable labels include radioisotopes, iodine (¹²⁵I, ¹²¹I), carbon (¹⁴C), sulphur (³⁵S), tritium (³H), indium (¹¹²In), and technetium (99mTc), fluorescent labels, such as fluorescein and rhodamine, and biotin. Immunoenzymatic interactions can be visualized using different enzymes such as peroxidase, alkaline phosphatase, or different chromogens such as DAB, AEC or Fast Red. The labeled antibody or antibody fragment will preferentially accumulate at the location of cells that contain a biomarker. The labeled antibody or variant thereof, e.g., antibody fragment, can then be detected using known techniques.

In certain non-limiting embodiments, agents that specifically bind to a protein other than antibodies are used, such as peptides. Peptides that specifically bind can be identified by any means known in the art, e.g., peptide phage display libraries. Generally, an agent that is capable of detecting a protein, such that the presence of the protein is detected and/or quantitated, can be used.

In addition, a protein can be detected using Mass Spectrometry such as MALDI/TOF (time-of-flight), SELDI/TOF, liquid chromatography-mass spectrometry (LC-MS), gas chromatography-mass spectrometry (GC-MS), high-performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See, for example, U.S. Patent Application Nos. 2003/0199001, 2003/0134304, 2003/0077616, which are herein incorporated by reference in their entireties.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait at al., Science 262:89-92 (1993); Keough at al., Proc. Natl. Acad. Sci. USA. 96:7131-6 (1999); reviewed in Bergman, EXS 88:133-44 (2000).

Detection of the presence of nucleic acid and/or protein will typically involve the detection of signal intensity. This, in turn, can reflect the quantity and character of a polypeptide bound to the substrate. For example, in certain embodiments, the signal strength of peak values from spectra of a first sample and a second sample can be compared (e.g., visually or by computer analysis) to determine the relative amounts of a particular biomarker. Software programs such as the Biomarker Wizard program (Ciphergen Biosystems, Inc., Fremont, Calif.) can be used to aid in analyzing mass spectra.

Additional methods for determining nucleic acid and/or protein expression in samples are described, for example, in U.S. Pat. Nos. 6,271,002; 6,218,122; 6,218,114; and 6,004,755; and in Wang et al, J. Clin. Oncol., 22(9): 1564-1671 (2004); and Schena et al., Science, 270:467-470 (1995); all of which are incorporated herein by reference in their entireties.

Methods of Treatment

The present disclosure relates to methods for preventing and/or treating a disease and/or disorder of a subject. In certain embodiments, the disease can be cancer in the subject. In certain embodiments, the disease can be the presence of cancer in the subject.

The present disclosure provides methods for preventing and/or treating a disease, e.g., cancer, in a subject by reducing the expression, activity and/or function of a marker of the transition of ASCs to COL11A1-expressing CAFs. The marker can include the disclosed lncRNAs, miRNAs, and additional targets. In certain embodiments, methods for preventing and/or treating a disease, e.g., cancer, in a subject include reducing the expression, activity and/or function of the disclosed lncRNAs, miRNAs, and additional targets.

In certain non-limiting embodiments, the present disclosure provides for preventing and/or treating a subject that has a disease, e.g., cancer. For example, but not by way of limitation, the method can include administering a therapeutically effective amount of the disclosed inhibitor of the marker of the transition of ASCs to COL11A1-expressing CAFs. In certain embodiments, administration of the inhibitor can inhibit the proliferation and/or survival of cancer cells in the subject.

In certain embodiments, the present disclosure provides a method for lengthening the period of survival of a subject having a disease, e.g., cancer. For example, but not by way of limitation, one or more of the disclosed inhibitors can be administered to a subject and prolong the survival of the subject relative to a control subject or control subject population not receiving the disclosed treatment. In certain embodiments, the period of survival is extended at least about 10 percent, at least about 25 percent, at least about 30 percent, at least about 50 percent, at least about 60 percent or at least about 70 percent. In certain embodiments, the period of survival is extended by about 1 month, about 2 months, about 4 months, about 6 months, about 8 months, about 10 months, about 12 months, about 14 months, about 18 months, about 20 months, about 2 years, about 3 years, about 5 years or more. In certain embodiments, the disclosed inhibitors can prolong the remission of cancer in the subject relative to a control subject or control subject population not receiving the disclosed treatment.

In certain embodiments, the present disclosure provides methods for producing an anti-cancer effect in a subject. For example, but not by way of limitation, the method for producing an anti-cancer effect includes administering to a subject having cancer a therapeutically effective amount of one or more of the disclosed inhibitors to produce an anti-cancer effect in the subject. In certain embodiments, the anti-cancer effect is selected from the group consisting of a reduction in aggregate cancer cell mass, a reduction in cancer cell growth rate, a reduction in cancer cell proliferation, a reduction in tumor mass, a reduction in tumor volume, a reduction in cancer cell proliferation, a reduction in cancer growth rate, a reduction in cancer metastasis, and combinations thereof. In certain embodiments, the anti-cancer effect is a reduction in the number of cancer cells. In certain embodiments, the anti-cancer effect is a reduction in tumor size and/or a reduction in the rate of tumor growth. In certain embodiments, the anti-cancer effect is a reduction in the aggregate cancer cell burden. In certain embodiments, the anti-cancer effect is a reduction in the rate of cell proliferation and/or an increase in the rate of cell death. In certain embodiments, the anti-cancer effect is a prolongation of the survival of the subject. In certain embodiments, the anti-cancer effect is a prolongation in the interval until relapse relative to a control subject or control subject population not receiving the disclosed treatment.

In certain embodiments, the present disclosure provides methods for reducing tumor growth in a subject. For example, but not by way of limitation, the method for reducing tumor growth includes administering to a subject a therapeutically effective amount of one or more of the disclosed inhibitors. In certain embodiments, tumor growth can be inhibited or reduced by decreasing expression, activity or function of the disclosed lncRNAs, miRNAs, and additional targets. As shown in the Examples described herein, lncRNAs, miRNAs, and additional targets are involved in various cancers.

Methods disclosed herein can be used for treating any suitable cancers. Non-limiting examples of cancers that can be treated accordingly the presently disclosed methods include carcinomas as well as any other cancer type, in which tumor cells are in the vicinity of adipose tissue at some stage of the disease, e.g., breast cancer, pancreatic cancer, and ovarian cancer.

In certain embodiments, a method for treating a subject having cancer can include determining the expression level of the disclosed biomarkers (e.g., the disclosed lncRNAs, miRNAs, or additional targets) in a sample of cancer, where if the expression/activity level of the disclosed biomarkers is increased compared to a reference level, then administering to the subject a therapeutically effective amount of an inhibitor disclosed herein. Any suitable detecting methods known in the art and disclosed herein can be used with the presently disclosed subject matter to detect the expression level of the disclosed biomarkers in the sample of the subject.

In certain embodiments, the dosage administered varies depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. In addition, it is to be understood that, for any particular subject, specific dosage regimes should be adjusted over time according to the individual need and the professional judgment of the person administering or supervising the administration of the inhibitor. For example, the dosage of the inhibitor can be increased if the lower dose does not provide sufficient activity in the treatment of a disease or condition described herein (e.g., cancer). Alternatively, the dosage of the inhibitor can be decreased if the disease (e.g., cancer) is reduced, no longer detectable or eliminated.

In certain embodiments, the disclosed inhibitors can be administered to the subject in a single dose or divided doses. In certain embodiments, the disclosed inhibitors can be administered to the subject once a day, twice a day, once a week, twice a week, three times a week, four times a week, five times a week, six times a week, once every two weeks, once a month, twice a month, once every other month or once every third month.

In certain embodiments, the duration of the disclosed treatment can be between about one week to about two years. In certain embodiments, the duration of the disclosed treatment is at least about 1 week, at least about 2 weeks, at least about 3 weeks, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, at least about 13 months, at least about 14 months, at least about 15 months, at least about 16 months, at least about 17 months, at least about 18 months, at least about 19 months, at least about 20 months, at least about 21 months, at least about 22 months, at least about 23 months, or at least about 24 months. In certain embodiments, the duration of the disclosed treatment is at most about 1 week, at most about 2 weeks, at most about 3 weeks, at most about 1 month, at most about 2 months, at most about 3 months, at most about 4 months, at most about 5 months, at most about 6 months, at most about 7 months, at most about 8 months, at most about 9 months, at most about 10 months, at most about 11 months, at most about 12 months, at most about 13 months, at most about 14 months, at most about 15 months, at most about 16 months, at most about 17 months, at most about 18 months, at most about 19 months, at most about 20 months, at most about 21 months, at most about 22 months, at most about 23 months, or at most about 24 months. In certain embodiments, the duration of the disclosed inhibitor treatment is at most 24 months or 2 years. In certain embodiments, the inhibitor can be administered until cancer is no longer detectable.

In certain embodiments, the disclosed inhibitors can be cyclically administered to a subject. Cycling therapy involves the administration of the inhibitors for a period of time, followed by a rest for a period of time, and repeating this sequential administration. Cycling therapy can reduce the development of resistance to one or more of the therapies, avoid or reduce the side effects of one of the therapies, and/or improve the efficacy of the treatment. In certain embodiments, the treatment stops after one cycle because the subject is intolerable to the adverse effects and toxicities associated with the disclosed inhibitors.

In certain embodiments, the number of cycles is from about one to about twenty-four cycles. In certain embodiments, the number of cycles is more than twenty-four cycles. In certain embodiments, the number of cycles is about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, or about 24. In certain embodiments, the duration of a cycle is from about 21 to about 30 days. In certain embodiments, the duration of a cycle is about 21, about 22, about 23, about 24, about 25, about 26, about 27, about 28, about 29, or about 30 days. In certain embodiments, the duration of a cycle is about 27 days, about 28 days, about 29, or about 30 days. In certain embodiments, the number of cycles is about twenty-four cycles.

In certain embodiments, each cycle is followed by a rest period, where the disclosed inhibitors are not administered to the subject. In certain embodiments, the rest period is from about two weeks to about six weeks, from three weeks to about five weeks, from about four weeks to about six weeks. In certain embodiments, the rest period is about three weeks, about four weeks, about five weeks, or about six weeks. The present disclosure further allows the frequency, number, and length of dosing cycles and rest periods to be adjusted.

In certain embodiments, the inhibitors and agonists disclosed herein can be used alone or in combination with one or more agents, e.g., anti-cancer agents. For example, but not by way of limitation, methods of the present disclosure can include administering one or more inhibitors and one or more agents, e.g., anti-cancer agents. “In combination with,” as used herein, means that the disclosed inhibitor and the one or more agents, e.g., anti-cancer agents, are administered to a subject as part of a treatment regimen or plan. In certain embodiments, being used in combination does not require that the inhibitor and one or more agents, e.g., anti-cancer agents, are physically combined prior to administration, administered by the same route or that they be administered over the same time frame. In certain embodiments, the agent, e.g., anti-cancer agent, is administered before an inhibitor or agonist. In certain embodiments, the agent, e.g., anti-cancer agent, is administered after an inhibitor or agonist. In certain embodiments, the agent, e.g., anti-cancer agent, is administered simultaneously with an inhibitor or agonist. In certain embodiments, the disclosed inhibitor can be administered in combination with one or more agents, e.g., anti-cancer agents.

In certain embodiments, one or more agents can be an anti-cancer agent. Non-limiting exemplary anti-cancer agents include, but are not limited to, chemotherapeutic agents, radiotherapeutic agents, cytokines, anti-angiogenic agents, apoptosis-inducing agents, anti-cancer antibodies, a targeted drug, and/or agents which promote the activity of the immune system, including but not limited to cytokines such as but not limited to interleukin 2, interferon, an anti-CTLA4 antibody, an anti-PD-1 antibody and/or an anti-PD-L1 antibody, and checkpoint inhibitors. In certain embodiments, the anti-cancer agent can be a taxane, a platinum-based agent, an anthracycline, an anthraquinone, an alkylating agent, a HER2 targeting therapy, vinorelbine, a nucleoside analog, ixabepilone, eribulin, cytarabine, a hormonal therapy, methotrexate, capecitabine, lapatinib, 5-FU, vincristine, etoposide or any combination thereof. In certain embodiments, the anti-cancer agent can be radiation therapy. Other non-limiting exemplary anti-cancer agents that can be used with the presently disclosed subject matter include tumor-antigen-based vaccines and chimeric antigen receptor T-cells.

In certain embodiments, the one or more agents can be an agent that is the standard of care for a disease. For example, but not by way of limitation, the one or more agents can be the standard of care for treating diabetes, e.g., insulin, an insulin analog, metformin or similar diabetes agents, sulfonylureas, meglitinides, thiazolidinediones, DPP-4 inhibitors, GLP-1 receptor agonists and SGLT2 inhibitors.

### IV. Pharmaceutical Compositions

The present disclosure further provides pharmaceutical compositions comprising one or more of the disclosed cancer transition inhibitors for use in treating a disease in a subject. In certain embodiments, the disease can be cancer. For example, but not by way of limitation, the inhibitor can be selected from the group consisting of the disclosed lncRNA inhibitor, the disclosed miRNA inhibitor, the disclosed additional target inhibitor, or combinations thereof.

In certain embodiments, a pharmaceutical composition of the present disclosure includes the disclosed cancer transition inhibitor and a pharmaceutically acceptable carrier. Suitable pharmaceutically acceptable carriers that can be used with the presently disclosed subject matter have the characteristics of not interfering with the effectiveness of the biological activity of the active ingredients, e.g., disclosed inhibitors/anti-cancer agents, and that is not toxic to the patient to whom it is administered. Non-limiting examples of suitable pharmaceutical carriers include phosphate-buffered saline solutions, water, emulsions, such as oil/water emulsions, various types of wetting agents, and sterile solutions. Additional non-limiting examples of pharmaceutically acceptable carriers include gels, bioabsorbable matrix materials, implantation elements containing the inhibitor and/or any other suitable vehicle, delivery or dispensing means or material. Such pharmaceutically acceptable carriers can be formulated by conventional methods and can be administered to the subject. In certain embodiments, the pharmaceutical acceptable carriers can include buffers such as phosphate, citrate, and other organic acids; antioxidants including ascorbic acid and methionine; preservatives (such as, but not limited to, octadecyl-dimethyl benzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol and m-cresol); low molecular weight (less than about 10 residues) polypeptides; proteins, such as serum albumin, gelatin or immunoglobulins; hydrophilic polymers such as polyvinylpyrrolidone; amino acids such as glycine, glutamine, asparagine, histidine, arginine or lysine; monosaccharides, disaccharides, and other carbohydrates including glucose, mannose or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). In certain embodiments, the suitable pharmaceutically acceptable carriers can include one or more of water, saline, phosphate-buffered saline, dextrose, glycerol, ethanol or combinations thereof.

In certain non-limiting embodiments, the pharmaceutical compositions of the present disclosure can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions and the like, for oral or nasal ingestion by a patient to be treated. In certain embodiments, the pharmaceutical composition is formulated as a capsule. In certain embodiments, the pharmaceutical composition can be a solid dosage form. In certain embodiments, the tablet can be an immediate-release tablet. Alternatively or additionally, the tablet can be an extended or controlled release tablet. In certain embodiments, the solid dosage can include both an immediate release portion and an extended or controlled release portion.

In certain embodiments, the pharmaceutical compositions of the present disclosure can be formulated using pharmaceutically acceptable carriers well known in the art that are suitable for parenteral administration. The terms “parenteral administration” and “administered parenterally,” as used herein, refers to modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. For example, and not by way of limitation, pharmaceutical compositions of the present disclosure can be administered to the patient intravenously in a pharmaceutically acceptable carrier such as physiological saline. In certain embodiments, the present disclosure provides a parenteral pharmaceutical composition comprising inhibitors disclosed herein.

In certain embodiments, the pharmaceutical compositions suitable for use in the presently disclosed subject matter can include compositions where the active ingredients, e.g., the disclosed cancer transition inhibitor, are contained in a therapeutically effective amount. The therapeutically effective amount of an active ingredient can vary depending on the active ingredient, compositions used, cancer and its severity, and the age, weight, etc., of the subject to be treated. In certain embodiments, a subject can receive a therapeutically effective amount of the disclosed inhibitors in single or multiple administrations of one or more compositions, which can depend on the dosage and frequency as required and tolerated by the patient.

In certain embodiments, pharmaceutical compositions of the present disclosure can include or more anti-cancer agents or be administered in combination with one or more additional anti-cancer agents. Non-limiting exemplary anti-cancer agents include, but are not limited to, chemotherapeutic agents, radiotherapeutic agents, cytokines, anti-angiogenic agents, apoptosis-inducing agents, anti-cancer antibodies, a targeted drug, agents which promote the activity of the immune system, and checkpoint inhibitors. Other non-limiting exemplary anti-cancer agents that can be used with the presently disclosed subject matter include tumor-antigen-based vaccines and chimeric antigen receptor T-cells.

The following examples are offered to more fully illustrate the disclosure but are not to be construed as limiting the scope thereof.

### Example 1: Single-Cell Analysis Reveals the Pan-Cancer Invasiveness-Associated Transition of Adipose-Derived Stromal Cells into COL11A1-Expressing Cancer-Associated Fibroblasts

Computational analysis of rich gene expression data at the single-cell level from cancer biopsies can lead to biological discoveries about the nature of the disease. Using a computational methodology that identifies the gene expression profile of the dominant cell population for a particular cell type in the microenvironment of tumors, a remarkably continuous modification of this profile among patients, corresponding to a cellular transition, was observed. Specifically, the starting point of this transition has a unique characteristic signature corresponding to cells that are naturally residing in normal adipose tissue. The endpoint of the transition has another characteristic signature corresponding to a particular type of cancer-associated fibroblasts with the prominent expression of gene COL11A1, which has been found strongly associated with invasiveness, metastasis and resistance to therapy in multiple cancer types. The disclosed results provide an explanation to the well-known fact that the adipose tissue contributes to cancer progression, shedding light on the biological mechanism by which tumor cells interact with the adipose microenvironment. The disclosed subject matter provides a detailed description of the changing profile during the transition, identifying associated genes as potential targets for pan-cancer therapeutics inhibiting the underlying mechanism.

Using computational analysis of rich single-cell datasets from many patients, the nature and origin of a particular type of cancer-associated fibroblasts (CAFs) that has been found to be strongly associated with invasiveness, metastasis, resistance to therapy, and poor prognosis, in multiple cancer types were identified. These fibroblasts can be identified by their characteristic signature with the prominent presence of collagen COL11A1 and several other co-expressed genes such as THBS2 and INHBA. As described below, the disclosed subject provides techniques for assessing the dynamic changes in gene expression of cells associated with lineages, such as trajectory inference, as well as complementary computational approaches with novel application in single-cell data analysis. These techniques allowed the precise identification of the expression profile of the origin of the underlying cellular transition as a particular cell type of adipose-derived stromal/stem cells (ASCs). The presence of those ASCs was validated as naturally occurring, by applying the same computational methods in other available datasets of normal adipose tissue.

These CAFs were first identified in 2010 [1] by their cancer stage-associated signature. Specifically, a COL11A1/INHBA/THBS2-expressing gene signature was found to be present only after a particular staging threshold, different in each cancer type, was reached. For example, it only appeared in ovarian cancer of at least stage III; in colon cancer of at least stage II; and in breast cancer of at least invasive stage I (but not in carcinoma in situ). The striking consistency of that signature was observed across cancer types, which was obvious at that time from bulk microarray data. For example, Table 1 shows the top 15 genes ranked in terms of fold change for three different cancer types (breast [2], ovarian [3], pancreatic [4]) using data provided in papers published independently. The breast cancer data compare invasive ductal carcinoma with ductal carcinoma in situ (supplementary data 3 of the paper [2]; “up in IDC”); the ovarian cancer data compare metastatic tissue in the omentum with primary tumor (Table 2 of the paper [3]), and the pancreatic data compare whole tumor tissue with normal pancreatic tissue (Table 1 of the paper [4]).

TABLE 1
Top 15 ranked genes in terms of fold change (FC) for three different
cancer types revealing the signature of the COL11A1-expressing
cancer-associated fibroblasts. Multiple entries of the same gene
are not shown (keeping the one that appears first) and dashes.

| | | | | | Pancreatic | |
|---|---|---|---|---|---|---|
| | Breast | | Ovarian | | | Log2 |
| Rank | Gene | FC | Gene | FC | Gene | FC |
| 1 | COL11A1* | 6.5 | COL11A1* | 8.23 | INHBA* | 5.15 |
| 2 | COL10A1** | 4.07 | COL1A1** | 5.67 | COL10A1** | 5 |
| 3 | MFAP5** | 3.73 | TIMP3 | 5.52 | POSTN** | 4.92 |
| 4 | LRRC15 | 3.61 | FN1** | 5.4 | SULF1** | 4.63 |
| 5 | INHBA* | 3.44 | INHBA* | 4.94 | COL8A1 | 4.6 |
| 6 | FBN1** | 3.43 | EFEMP1 | 4.86 | COL11A1* | 4.4 |
| 7 | SULF1** | 3.35 | DSPG3 | 4.36 | CTHRC1 | 4.38 |
| 8 | GREM1 | 3.35 | COL5A2* | 4.07 | COL1A1** | 4.12 |
| 9 | COL5A2* | 3.22 | LOX** | 4.03 | THBS2* | 3.97 |
| 10 | LOX** | 3.22 | MFAP5** | 4.01 | HNT | 3.9 |
| 11 | COL5A1** | 3.08 | POSTN** | 3.97 | CSPG2 | 3.87 |
| 12 | THBS2* | 2.99 | COL5A1** | 3.95 | WISP1 | 3.8 |
| 13 | LAMB1 | 2.97 | THBS2* | 3.91 | FN1** | 3.69 |
| 14 | FAP** | 2.96 | FBN1** | 3.9 | COMP | 3.53 |
| 15 | SPOCK | 2.91 | FAP** | 3.84 | COL5A2* | 3.38 |

*shows genes shared in all three cancer types.
**shows genes appearing twice.

The four genes COL11A1, INHBA, THBS2, and COL5A2 appear among the top 15 in all three sets (P=6×10-23 by multi-set intersection test [5]). The actual P-value is much lower than that because, in addition to the above overlap, ten additional genes (COL10A1, COL1A1, COL5A1, PAP, PBN1, PN1, LOX, MPAP5, POSTN, SULP1) appear among the top 15 in at least two of the three sets (and are highly ranked in all three sets anyway). This similarity demonstrates that the signature is well-defined and associated with a universal biological mechanism in cancer.

Gene COL11A1 serves as a proxy of the full signature, in the sense that it is the only gene from which all other genes of the signature are consistently top-ranked in terms of the correlation of their expression with that of COL11A1. Accordingly, a COL11A1-correlated pan-cancer gene signature, listed in table 4 of [1], which was deposited in the Molecular Signatures Database (MSigDB), was identified. Those CAFs were referred to as MAFs (“metastasis-associated fibroblasts”), because their presence suggests that metastasis is imminent. To avoid any inaccurate interpretation of the term as implying that such fibroblasts are markers of metastasis that has occurred already, here they are referred as “COL11A1-expressing CAFs.”

Since then, many research results have been published connecting one of the genes COL11A1, INHBA, THBS2, with poor prognosis, invasiveness, metastasis, or resistance to therapy, in various cancer types [6-15].

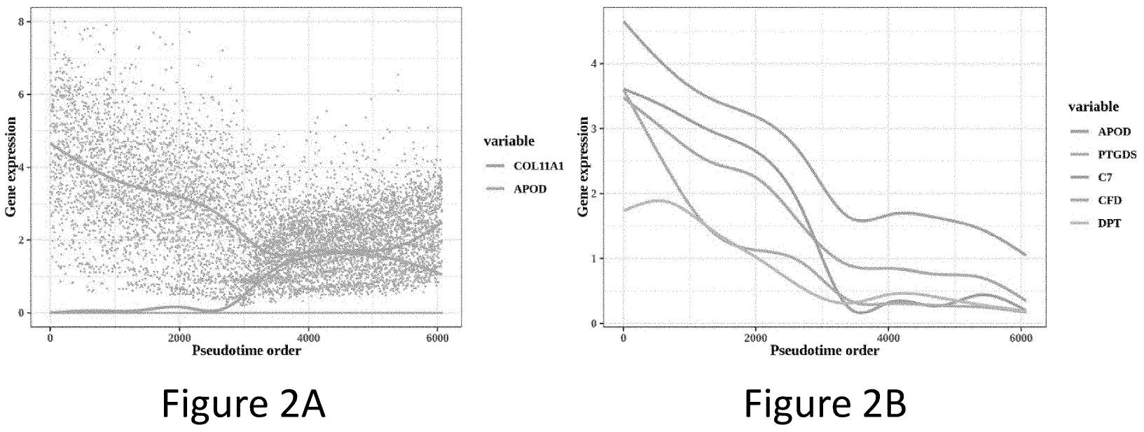

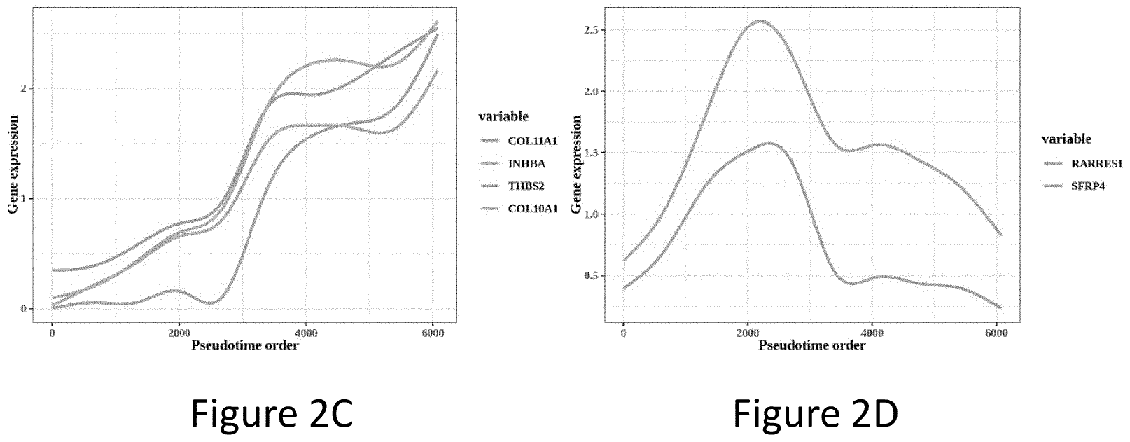

Furthermore, several designated tumor subtypes were identified in individual cancer types as a result of the presence of those pan-cancer CAFs. For example, the top 15 genes distinguishing the ovarian “mesenchymal subtype” according to [16] are POSTN, COL11A1, THBS2, COL5A2, ASPN, PAP, MMP13, VCAN, LUM, COL10A1, CTSK, COMP, CXCL14, PABP4, INHBA. Similarly, the 24 characterizing genes of the “activated stroma subtype” of pancreatic cancer in FIG. 2 of [17] are SPARC, COL1A2, COL3A1, POSTN, COL5A2, COL1A1, THBS2, PN1, COL10A1, COL5A1, SPRP2, CDH11, CTHRC1, PNDC1, SULP1, PAP, LUM, COL11A1, ITGA11, MMP11, INHBA, VCAN, GREM1, COMP. In both of these examples, these gene lists are clearly due to the presence of the COL11A1/INHBA/THBS2-expressing CAFs, and therefore, these are not cancer-type specific subtype signatures.

To computationally investigate the origin of those CAFs, the analysis of rich datasets from single-cell RNA sequencing (scRNA-seq) provides unique opportunities for tracking the trajectories of cell differentiation lineages. There are several single-cell trajectory inference methods [18] performing “trajectory inference analysis,” ordering cells along a trajectory based on similarities in expression patterns.

In particular, one exceptionally rich dataset [19] was identified from pancreatic ductal adenocarcinoma, containing gene expression profiles from 24 tumor samples and 11 normal control samples. Several among the 24 tumor samples contained populations of cells strongly co-expressing COL11A1, THBS2 and INHBA, while none of the normal samples contained such cells. The prominence of this co-expression signature varied significantly among the tumor samples, having only hints of their presence in some of them, suggesting that the corresponding patients were at various stages of the generation of COL11A1-expressing CAFs. This provides an opportunity to perform additional complementary computational analysis by comparing the prevalent fibroblastic cell populations across the tumor samples and comparing them with those in the normal samples.

Therefore, the attractor analysis was used in a novel manner for the analysis of rich scRNA-seq data. The unsupervised attractor algorithm [20] iteratively finds co-expression signatures converging to “attractor metagenes” pointing to the core (“heart”) of co-expression. Each attractor metagene is defined by a ranked set of genes along with scores determining their corresponding strengths within the signature, so the top-ranked genes are the most representative of the signature. The attractor algorithm has previously been used successfully for identifying features useful for breast cancer prognosis [21,22]. When applied on single-cell data from a sample, it identifies the gene expression profiles of the dominant cell populations in the sample, and the algorithm is designed to ensure that all the top-ranked genes are co-expressed in the same cells. The purpose of the attractor algorithm is not to classify cells into mutually exclusive subsets. Instead, it identifies the genes at the core of co-expression signatures representing cellular populations from single-cell data, and it provides information that cannot be deduced with traditional clustering methods (see Discussion).

When the attractor algorithm was separately applied in each of the normal samples, a set of nearly identical attractor signatures was identified, corresponding to a type of adipose-derived stromal/stem cells (ASCs) naturally present in the stromal vascular fraction (SVF) of normal adipose tissue, expressing a unique characteristic signature containing fibroblastic markers such as LUM and DCN as well as adipose-related genes, such as APOD, CFD and MGP.

When the algorithm was applied in each of the tumor samples, a set of signatures that were changing in a remarkably continuous manner across the samples, some of them being very similar to those of the normal samples, while others are similar to the COL11A1-based signature, was found. This suggests that the signatures undergo a gradual change as the transition proceeds, starting from the state of the normal ASCs and passing through a continuum of intermediate states. These results were consistent with those found by applying trajectory inference analysis, but they provided additional significant information based on their unique capabilities. Accordingly, this method demonstrated that there is a continuous “ASC to COL11A1-expressing CAF transition.”

This finding explains the stage association of the COL11A1-expressing signature as resulting from the interaction of tumor cells with the adipose microenvironment: Indeed, adipose tissue is encountered when ovarian cancer cells reach the omentum (stage III); after colon cancer has grown outside the colon (stage II); and in breast cancer from the beginning of the spread (stage I, but not in situ stage 0).

Finally, the results were validated in other cancer types (head and neck, ovarian, lung, breast), suggesting the pan-cancer nature of the ASC to COL11A1-expressing CAF transition.

Datasets availability: The pancreatic dataset [19] was downloaded from the Genome Sequence Archive with accession number CRA001160. The four validation datasets of other cancer types are also publicly available: HNSCC [36] (GSE103322), ovarian [37] (GSE118828), lung cancer [38] (E-MTAB-6149 and E-MTAB-6653), breast cancer [39] (GSE118389).

Samples from lymph nodes were excluded. The numbers of patients included in these datasets are 35 (PDAC), 18 (HNSCC), 9 (ovarian), 5 (lung), and 6 (breast).

Data processing and cell identification: The Seurat R toolkit [55] was selected for data processing and cell identification. Seurat implements the entire clustering workflow and has an advantage in speed and scalability to analyze large datasets [56]. The Seurat (v3.1.4) was applied to process the gene expression matrix and characterize the cell type identity for each scRNA-seq dataset. The count matrix was normalized and log-transformed by using the NormalizeData function. The 2,000 most variable features were selected, and then principal component analysis (PCA) was performed, followed by applying an unsupervised graph-based clustering approach. Default parameter settings were selected in all the above steps except that the resolution parameter in the FindCluster function is set to 1.0 to increase the granularity of downstream clustering. To identify differentially expressed genes for each cluster, the FindMarkers function was used. To characterize the identity of mesenchymal cells in each dataset, the expression of known markers were used: LUM, DCN, COL1A1 for fibroblasts, and RGS5, ACTA2, PDGFRB and ADIRF for pericytes.

For the smaller-size datasets (ovarian, breast), clustering was performed once on all cells for mesenchymal cell identification. For datasets of larger size (PDAC, HNSCC, lung), the two-step clustering was used to ensure accuracy: The first step was initial clustering within individual samples. Then, samples with very few (<20) detected fibroblasts and pooled the mesenchymal cells of the remaining samples together for a second clustering, which resulted in the final set of mesenchymal cells for the dataset, were excluded. For the PDAC dataset, an additional step was performed to remove low-quality cells, by retaining cells for which at least one of the corresponding markers had expression levels ˜3.

Mutual information: Mutual information (MI) is a general measure of the association between two random variables [57]. A spline-based estimator [58] was used to estimate MI values and normalized, so the maximum possible value is 1. The MI value is clipped to zero if the Pearson correlation between the two variables is negative. The details of the estimation method are described in the paper introducing the attractor algorithm [20]. The getMI or getAllMIWz function implemented in the cafr R package was used with parameter negateMI=TRUE.

Attractor-based analysis: The attractor algorithm was first proposed for identifying co-expression signatures from bulk expression values in samples [20]. The attractor algorithm was used for the first time for the purpose of scrutinizing cell populations in single-cell data. Compared to conventional single-cell methods, the attractor algorithm features the unique capability of discovering precise profiles of cell populations, which other methods cannot achieve.

Briefly, the algorithm iteratively finds mutually associated genes from an expression matrix, converging to the core of the co-expression mechanism. The association measure used is the normalized mutual information, which captures the general relationships (including nonlinear effects) between variables. Using the expression vector corresponding to a seed gene as input, the algorithm converges to an “attractor” in the form of a list of ranked genes, together with scores (ranging from 0 to 1) for each of these genes measuring the strength of the membership of that gene in the signature. It has a characteristic property that using different “attractee” genes belonging to a co-expression signature as seeds leads to the identical attractor.

The attractor algorithm had previously been used to find co-expression signatures in bulk gene expression data, in which case a converged attractor can represent a mixture of contributions from distinct cell subpopulations. When using single-cell data, however, the characteristic genes of particular distinct subpopulations will have high expression values only in the cells from those subpopulations and low values in other cells. These genes can have pair-wise positive and large correlations, and therefore they will be highly ranked in attractor signatures representing such individual subpopulations. On the other hand, two characteristic marker genes belonging to two different distinct subpopulations can have reverse-associated expression values across those cells, which can contribute negatively to the overall correlation between these two genes. Only if two genes are co-expressed across individual cells, they appear highly ranked in the same attractor.

For a single dataset, the attractor finding algorithm was applied using the findAttractor function implemented in the cafr (v0.312) R package [20] with the general fibroblastic marker gene LUM as seed. Identical results in all samples can be found, with very rare exceptions, if other fibroblastic markers, such as DCN, are used. The exponent (a) was set to different values for scRNA-seq datasets profiled from different protocols. For the analysis of UMI based (e.g. 10×) and full-length-based (e.g. Smart-seq2) datasets, a=3 and a=5 were used, respectively. To find the consensus attractor for multiple datasets, the consensus version of the attractor was applied finding algorithm as described in [59]. In the consensus version, the association measures between genes are evaluated as the weighted median of the corresponding measures taken from the individual datasets. The weights are proportional to the number of samples included in each individual dataset in a log scale.

Trajectory inference (TI) analysis: the Slingshot [35] method was selected for TI analysis based on its robustness and suggestions made by the benchmarking pipeline dynverse [18]. The raw counts were used as input and followed the Slingshot lineage analysis workflow (v1.4.0). To begin this process, Slingshot chose robustly expressed genes if it has at least 10 cells that have at least 1 read for each. After gene filtering, full quantile normalization was performed. Following diffusion map dimensionality reduction, Gaussian mixture modeling was performed to classify cells, where the number of clusters in the Mclust function was set to 3 based on the fact that there were three clusters in the Seurat clustering results. The final step of lineage inference analysis used the slingshot wrapper function in an unsupervised manner. A cluster-based minimum spanning tree was subjected to describe the lineage. After analyzing the global lineage structure, a generalized additive model (GAM) was fitted for pseudotime and computed P values. Genes were ranked by P values and variances. After running Slingshot, genes whose expression values significantly vary over the derived pseudotime were identified by using a GAM, allowing us to detect non-linear patterns in gene expression.

Statistical analysis: P-value evaluation for overlapping genes from different sets. The hypergeometric test was applied for evaluating the significance of genes shared by different sets. If there are two sets to compare, the phyper R function was used. If there are more than two sets to compare, the multi-set intersection test [5] was used by applying the cpsets function implemented in the SuperExactTest R package. Regarding the background universe size of genes, the total number of genes analyzed in the specific expression matrix was used. In the case of comparing sets coming from different studies, 20,000 was used as the universe size.

Differential expression analysis: A Wilcoxon Rank Sum test was used by applying the FindMarkers function in Seurat to identify the differentially expressed (DE) genes between fibroblasts of different groups. DE genes with log fold change >0.25 and Bonferroni adjusted value <0.1 are considered as significant. The positive and negative DE genes are ranked separately in terms of the absolute values of their log fold-change.

ASC to COL11A1-expressing CAF transition identified in pancreatic ductal adenocarcinoma (PDAC): The PDAC dataset [19] consists of 57,530 scRNA-seq profiles from 24 PDAC tumor samples (T1-T24) and 11 normal samples (N1-N11). To find the expression profile of the dominant fibroblastic population in each sample, the attractor algorithm was applied on the set of identified mesenchymal cells (Materials and Methods). All samples (11 normal and 23 tumor samples, excluding sample T20 as it did not contain identified fibroblasts) yielded strong co-expression signatures involving many genes with big overlap among them. Genes LUM, DCN, FBLN1, MMP2, SFRP2 and COL1A2 appear in the top 100 genes in at least 33 out of the 34 samples, revealing a strong similarity shared by all those fibroblastic expression profiles. This strong overlap is consistent with the continuous transition process, as described below.

Dominant fibroblastic population in the normal pancreatic samples is adipose-derived: There is a striking similarity among the attractor profiles of the eleven normal pancreatic samples, indicating that they represent a stable and normally occurring cell population. Specifically, there are 12 genes commonly shared among the top 30 genes in the attractors of at least ten of the eleven normal samples (Table 2), of which four genes are shared among all the samples (P=3×10-113 by multi-set intersection test [5]). In addition to fibroblastic markers, there are several strongly expressed adipose-related or stem-ness-related genes in the list, such as APOD, CXCL12, and DPT, revealing that they are ASCs. Consistently, Gene Set Enrichment Analysis (GSEA) of these 12 commonly shared genes identified the most significant enrichment (FDR q value=2.16×10-19) in the “BOQUEST_-STEM_CELL_UP” dataset of genes upregulated in stromal stem cells from adipose tissue versus the non-stem counterparts [23].

TABLE 2
Top 30 genes of the identified attractors for each pancreatic normal sample (N1-N11).

| Rank | N1 | N2 | N3 | N4 | N5 | N6 |
|---|---|---|---|---|---|---|
| 1 | DCN* | LUM* | LUM* | C7* | APOD* | LUM* |
| 2 | LUM* | DCN* | FBLN1* | FBLN5* | DPT* | DCN* |
| 3 | C7* | C7* | C7* | LUM* | FBLN5* | FBLN1* |
| 4 | FBLN1* | FBLN1* | PTGDS* | DCN* | PDGFRA* | ADH1B |
| 5 | MGP | APOD* | C1S | APOD* | CXCL12* | DPT* |
| 6 | C1S | MGP | DPT* | PTGDS* | LUM* | ABCA8 |
| 7 | CCDC80 | C1S | PDGFRA* | FBLN1* | COL6A3 | C3 |
| 8 | PTGDS* | DPT* | APOD* | C1R* | PTGDS* | APOD* |
| 9 | DPT* | CCDC80 | SFRP2* | DPT* | C7* | MMP2 |
| 10 | C1R* | PTGDS* | DCN* | SRPX | CCDC80 | C1S |
| 11 | APOD* | FBLN5* | CXCL12* | FMO2 | CFD | C7* |
| 12 | SEPP1 | SEPP1 | C1R* | SEPP1 | MRC2 | PTGDS* |
| 13 | FBLN5* | COL1A2 | COL6A3 | CXCL12* | FGF7 | SFRP2* |
| 14 | CXCL12* | SFRP2* | ADH1B | CYR61 | SFRP2* | FBLN5* |
| 15 | EFEMP1 | SRPX | SPON2 | SFRP2* | MARCKS | C1R* |
| 16 | COL1A2 | SERPINF1 | CFD | CLEC11A | LRP1 | CXCL12* |
| 17 | SFRP2* | OLFML3 | LAMA2 | PDGFRA* | FMO2 | CST3 |
| 18 | ALDH1A1 | CST3 | C3 | NR2F1 | NR2F1 | MGP |
| 19 | CFD | MEG3 | FBLN5* | C1S | TNXB | CCDC80 |
| 20 | COL6A | C1R* | ABCA8 | ABCA8 | DCN* | MRC2 |
| 21 | EMP1 | MFAP4 | LRP1 | CCDC80 | LOX | COL1A2 |
| 22 | PCOLCE | RARRES2 | SLIT2 | PTN | C1R* | CFD |
| 23 | C3 | PCOLCE | CFH | SERPINF1 | IGFBP3 | SPRY1 |
| 24 | SRPX | CFH | SRPX | SVEP1 | HEG1 | SMOC2 |
| 25 | SERPINF1 | CXCL12* | COL1A2 | CFD | RP11- | GSN |
| | | | | | 572C15.6 | |
| 26 | ANXA1 | FGF7 | BOC | LAMB1 | F3 | COL6A2 |
| 27 | CYR61 | PDGFRA* | FSTL1 | FTL | ADAMTSL3 | CFH |
| 28 | CST3 | COL6A3 | SVEP1 | ANTXR2 | STK17B | OLFML3 |
| 29 | RARRES2 | ALDH1A1 | ABCA9 | COL6A3 | EMP1 | PDGFRA* |
| 30 | PDGFRA* | SPRY1 | CYR61 | MGP | MPZL1 | PCOLCE |

| Rank | N7 | N8 | N9 | N10 | N11 |
|---|---|---|---|---|---|
| 1 | PTGDS* | C7* | DCN* | MMP2 | LUM* |
| 2 | APOD* | LUM* | LUM* | APOD* | DCN* |
| 3 | LUM* | DCN* | C7* | LUM* | FBLN1* |
| 4 | FBLN1* | APOD* | FBLN1* | EFEMP1 | SFRP2* |
| 5 | C7* | FBLN1* | APOD* | CTSK | CFD |
| 6 | ADH1B | SFRP2* | SFRP2* | SFRP2* | APOD* |
| 7 | DPT* | PTGDS* | SERPINF1 | PLTP | MGP |
| 8 | COL6A3 | CCDC80 | PTGDS* | MGST1 | SERPINF1 |
| 9 | EFEMP1 | FBLN5* | GSN | LSP1 | CCDC80 |
| 10 | PDGFRA* | C1S | C1S | FBLN1* | C3 |
| 11 | CXCL12* | CXCL12* | SEPP1 | SPON2 | ADH1B |
| 12 | SCN7A | C3 | CCDC80 | PTGDS* | PTGDS* |
| 13 | MMP2 | CFD | DPT* | SVEP1 | C7* |
| 14 | MEG3 | C1R* | OLFML3 | CXCL12* | C1S |
| 15 | C1S | MGP | FBLN5* | SCN7A | CST3 |
| 16 | OLFML3 | CFH | C1R* | COL6A3 | C1R* |
| 17 | SVEP1 | COL6A3 | PTN | CCDC80 | CXCL14 |
| 18 | DCN* | SRPX | MGP | COLEC11 | MMP2 |
| 19 | SFRP2* | EFEMP1 | ALDH1A1 | PDGFRA* | GPNMB |
| 20 | MRC2 | SEPP1 | PDGFRA* | HBP1 | S100A4 |
| 21 | FBLN5* | PDGFRA* | COL6A2 | CYGB | DPT* |
| 22 | C3 | DPT* | CST3 | ARSK | MFAP4 |
| 23 | COL1A2 | CXCL14 | COL6A3 | SH3GL1 | COL6A2 |
| 24 | ABCA8 | ADH1B | CXCL14 | OAF | FBLN5* |
| 25 | SRPX | NEGR1 | C3 | BMP1 | SMOC2 |
| 26 | ACVRL1 | COL6A2 | CXCL12* | LAMA2 | ABCA8 |
| 27 | TIMP2 | BOC | MMP2 | GPC3 | FMO2 |
| 28 | LAMA2 | OLFML3 | PCOLCE | TMEM67 | RP11- |
| | | | | | 572C15.6 |
| 29 | DAB2 | EMP1 | IGF1 | C1R* | PCOLCE |
| 30 | NR2F1 | LAMA2 | ABCA8 | PLXDC1 | SEPP1 |

*shows 12 commonly shared genes in at least ten of the eleven normal samples.

To investigate the nature of this ASC population, results from single-cell analysis of general human adipose tissue were used [24]. The attractor algorithm was applied on the dataset with the single-cell expression profiles of all 26,350 cells taken from the SVF of normal adipose tissue from 25 samples and compared the identified attractor with the “consensus attractor” (Materials and Methods) of the 11 normal pancreatic samples, which represented the main state of the normal fibroblastic population (Table 3). There are 14 overlapping genes between the top 30 gene lists (P=10-33 by hypergeometric test), and most of the non-highlighted genes in each column are still ranked highly in the other column.

TABLE 3
Comparison of the attractors (top 30 genes) identified
in the SVF of normal adipose tissue (Dataset 1) and
in the normal pancreatic samples (Dataset 2).

| Rank | Dataset 1 | Dataset 2 |
|---|---|---|
| 1 | DCN* | LUM* |
| 2 | LUM* | DCN* |
| 3 | APOD* | FBLN1 |
| 4 | CFD* | C7 |
| 5 | CXCL14 | APOD* |
| 6 | MGP* | PTGDS |
| 7 | SERPINF1* | SFRP2 |
| 8 | GSN | C1S* |
| 9 | GPX3 | CCDC80* |
| 10 | MFAP4 | MGP* |
| 11 | PLAC9 | DPT* |
| 12 | S100A13 | CXCL12* |
| 13 | IGFBP6 | C1R |
| 14 | DPT* | FBLN5 |
| 15 | MFAP5 | C3 |
| 16 | FOS | PDGFRA |
| 17 | MGST1 | SRPX* |
| 18 | COL1A2* | COL6A3* |
| 19 | COL6A3* | ADH1B |
| 20 | LAPTM4A | CFD* |
| 21 | CXCL12* | OLFLM3 |
| 22 | WISP2 | SERPINF1* |
| 23 | SRPX* | MMP2* |
| 24 | JUN | CST3 |
| 25 | MMP2* | SEPP1 |
| 26 | COL6A2 | ABCA8 |
| 27 | C1S* | COL1A2* |
| 28 | CCDC80* | LAMB1 |
| 29 | EGR1 | SVEP1 |
| 30 | PCOLCE | MEG3 |

*represents common genes.

This extreme similarity of the two gene expression profiles indicates that they correspond to the same naturally occurring cell population. Furthermore, excluding the general fibroblastic markers LUM and DCN, gene APOD (Apolipoprotein D) has the highest average ranking in Table 3, and is top-ranked in the independently found SVF fibroblastic population of cluster VP4 (supplementary file 20) of [24]. Therefore, APOD was selected as the representative marker for the ASC population.

Establishing the presence of COL11A1-expressing CAFs in PDAC tumor samples: Because COL11A1 serves as proxy of the full signature [1], a reliable test for determining if a sample contains the COL11A1-expressing CAFs is to rank all genes in terms of their association, measured by mutual information (Materials and Methods), with COL11A1 and see if INHBA and THBS2 are top ranked. Indeed, this happens in several tumor samples, as shown in Table 4 for some of them (T23, T11, T6, T15, T18). For each sample, the shown genes are co-expressed in the same cells, because of the high correlations in a single-cell dataset.

TABLE 4
Ranked COL11A1-associated genes in five PDAC samples.

| Rank | T23 | MI | T11 | MI | T6 | MI | T15 | MI | T18 | MI |
|---|---|---|---|---|---|---|---|---|---|---|
| 1 | COL11A1* | 1 | COL11A1* | 1 | COL11A1* | 1 | COL11A1* | 1 | COL11A1* | 1 |
| 2 | COL10A1 | 0.3603 | CTHRC1 | 0.2434 | MFAP5 | 0.2353 | MFAP5 | 0.3198 | MFAP5 | 0.3408 |
| 3 | COL12A1 | 0.3383 | MFAP5 | 0.2357 | FNDC1 | 0.1997 | GJB2 | 0.2583 | SUGCT | 0.3379 |
| 4 | COL1A1 | 0.3187 | COL12A1 | 0.2345 | NTM | 0.1912 | COL10A1 | 0.2580 | COL10A1 | 0.2899 |
| 5 | THBS2* | 0.3167 | COL10A1 | 0.2238 | COL8A1 | 0.1877 | INHBA* | 0.2561 | C5orf46 | 0.2753 |
| 6 | COL1A2 | 0.3099 | C1QTNF3 | 0.2155 | TWIST1 | 0.1714 | C1QTNF3 | 0.2514 | PPAPDC1A | 0.2668 |
| 7 | COL5A2 | 0.3003 | THBS2* | 0.2123 | COL10A1 | 0.1619 | MATN3 | 0.2505 | NTM | 0.2649 |
| 8 | CTHRC1 | 0.2854 | COL1A2 | 0.2045 | THBS2* | 0.1559 | FNDC1 | 0.2503 | COL8A1 | 0.2534 |
| 9 | FN1 | 0.2781 | COL8A1 | 0.2018 | ITGA11 | 0.1556 | COL8A2 | 0.2411 | INHBA* | 0.2430 |
| 10 | COL3A1 | 0.2770 | AEBP1 | 0.2000 | PPAPDC1A | 0.1305 | COL1A1 | 0.2399 | FNDC1 | 0.2264 |
| 11 | INHBA* | 0.2746 | LUM | 0.1989 | DIO2 | 0.1298 | COL12A1 | 0.2351 | COL12A1 | 0.2194 |
| 12 | AEBP1 | 0.2688 | COL1A1 | 0.1985 | IGFL2 | 0.1178 | COL8A1 | 0.2325 | IGFL2 | 0.2153 |
| 13 | COL5A1 | 0.2626 | FNDC1 | 0.1963 | SUGCT | 0.1170 | THBS2* | 0.2292 | THBS2* | 0.2094 |
| 14 | VCAN | 0.2457 | SFRP2 | 0.1955 | ADAM12 | 0.1165 | NTM | 0.2257 | CTHRC1 | 0.2026 |
| 15 | MFAP5 | 0.2449 | GJB2 | 0.1879 | C1QTNF3 | 0.1165 | COL1A2 | 0.2220 | SULF1 | 0.2015 |
| 16 | MMP11 | 0.2360 | MATN3 | 0.1817 | ITGBL1 | 0.1109 | GREM1 | 0.2156 | COMP | 0.1926 |
| 17 | COL8A1 | 0.2357 | COL3A1 | 0.1740 | GREM1 | 0.1018 | FN1 | 0.2146 | STMN2 | 0.1926 |
| 18 | COL6A3 | 0.2339 | INHBA* | 0.1696 | P4HA3 | 0.1008 | IGFL2 | 0.2141 | WNT2 | 0.1925 |
| 19 | POSTN | 0.2316 | DCN | 0.1692 | INHBA* | 0.1002 | CXCL14 | 0.2112 | MMP11 | 0.1919 |
| 20 | MFAP2 | 0.2275 | CTGF | 0.1691 | COL5A1 | 0.0983 | ITGBL1 | 0.2048 | SPOCK1 | 0.1878 |

MI = Mutual Information.

Dominant fibroblastic populations in the tumor PDAC samples exhibits a continuous transition from ASCs to COL11A1-expressing CAFs: Based on the selection of APOD as a representative marker for the ASC population as described previously, the attractors of the PDAC tumor samples were rearranged in terms of descending order of the rank of APOD (Table 5) from left to right. There is a remarkable continuity in the shown expression profiles. The samples at the right side of the table include COL11A1 at increasingly high ranks. The intermediate tumor samples shown in the middle have cells expressing genes that are top-ranked in both the lists on the left as well as on the right. In other words, these cells are in a genuine intermediate state, rather than being a mixture of distinct subtypes.

TABLE 5
Rearranged PDAC tumor samples showing the continuously changing pattern of the signature profile. Columns
are sorted based on APOD rankings. Genes APOD and COL11A1 are highlighted in green and red, respectively.

| Rank | T2 | T13 | T14 | T19 | T3 | T10 | T15 |
|---|---|---|---|---|---|---|---|
| 1 | LUM | LUM | DCN | SFRP2 | MMP2 | PDGFRA | SFRP2 |
| 2 | APOD | APOD | APOD | APOD | LUM | HTRA3 | LUM |
| 3 | VCAN | DCN | LUM | LUM | APOD | DPT | DCN |
| 4 | SFRP4 | FBLN1 | SFRP4 | IGF1 | DCN | APOD | VCAN |
| 5 | SFRP2 | MMP2 | TSHZ2 | EFEMP1 | FBLN1 | MEG3 | APOD |
| 6 | MMP2 | SFRP4 | HTRA3 | PDGFRA | VCAN | OMD | FBLN1 |
| 7 | RARRES1 | SFRP2 | FBLN1 | OGN | FBLN5 | ITGBL1 | MMP2 |
| 8 | C3 | RARRES1 | MMP2 | SFRP4 | PDGFRA | PAPPA | COL6A3 |
| 9 | MEG3 | VCAN | COL6A3 | VCAN | SFRP2 | MRC2 | COL1A1 |
| 10 | HTRA3 | HTRA3 | VCAN | CTSK | C3 | LSAMP | COL1A2 |
| 11 | FBLN1 | ISLR | GPC3 | COL1A2 | MGP | CYP1B1 | ISLR |
| 12 | MGP | COL6A3 | CTGF | STEAP1 | EFEMP1 | COL10A1 | COL10A1 |
| 13 | DCN | SPON2 | SFRP2 | MMP2 | OMD | COL8A1 | CTHRC1 |
| 14 | CYP1B1 | CYP1B1 | C1S | CYP1B1 | RP11- | PDPN | CCDC80 |
| | | | | | 572C15.6 | | |
| 15 | COL1A2 | LXN | OMD | PTGDS | CCDC80 | CXCL14 | SFRP4 |
| 16 | MOXD1 | SERPINF1 | SPON2 | FBLN1 | IGF1 | MMP23B | CTSK |
| 17 | PTGDS | CTHRC1 | C3 | RARRES1 | TSHZ2 | ABCA9 | COL3A1 |
| 18 | FBLN5 | CTSK | F2R | DCN | ITM2A | LUM | S100A10 |
| 19 | PDGFRA | F2R | ANKH | COL3A1 | SFRP4 | PDGFRL | THBS2 |
| 20 | COL6A3 | FBLN5 | CTSK | MFAP5 | RARRES1 | STXBP6 | HTRA1 |
| 21 | CTHRC1 | COL1A2 | C1R | FBLN5 | COL8A1 | SVEP1 | SEMA3C |
| 22 | FAP | C7 | IGFBP3 | COL1A1 | OGN | BICC1 | FBLN2 |
| 23 | F2R | TMEM119 | MOXD1 | ISLR | C7 | ABCA6 | LRP1 |
| 24 | ISLR | EFEMP1 | CTHRC1 | C3 | DPT | MFAP2 | MRC2 |
| 25 | TIMP1 | MOXD1 | MEG3 | MEG3 | BICC1 | BNC2 | PDPN |
| 26 | C7 | CCDC80 | PDGFRA | CTHRC1 | CTHRC1 | WNT5A | MXRA5 |
| 27 | PHLDA3 | COL1A1 | FBLN5 | MOXD1 | PODN | CST3 | OMD |
| 28 | OMD | PLXDC2 | ITM2A | COL6A3 | COL6A3 | SERP2 | ITGBL1 |
| 29 | FBLN2 | PDGFRA | COL1A2 | MGP | CXCL14 | MOXD1 | RARRES2 |
| 30 | SCN7A | C3 | PTGDS | C7 | COL1A2 | ZFHX4 | FBN1 |
| 31 | EFEMP1 | COL10A1 | RARRES1 | CILP | ISLR | RARRES1 | PLXDC2 |
| 32 | COL10A1 | C1S | OLFML3 | COL8A1 | MEG3 | BOC | CXCL14 |
| 33 | SERPINF1 | PODN | OGN | THBS2 | CTSK | PODN | PDGFRA |
| 34 | BNC2 | LTBP2 | ITGBL1 | MRC2 | HSD11B1 | TMEM119 | MATN3 |
| 35 | CTSK | NPC2 | PTCH1 | NR2F1 | CTGF | PTGIS | COL8A1 |
| 36 | MRC2 | HSD11B1 | COL8A1 | ITGBL1 | C1S | OGN | NBL1 |
| 37 | TSHZ2 | MGP | BOC | TSHZ2 | COL10A1 | FAP | TMEM119 |
| 38 | LRP1 | DPT | SERPINF1 | STEAP2 | SERPINE2 | EFEMP1 | HTRA3 |
| 39 | SLIT2 | COL3A1 | MGP | MFAP2 | SERPINF1 | LAMA2 | FAP |
| 40 | COL1A1 | OMD | IGFBP6 | PDGFRL | LTBP2 | GSTM5 | MEG3 |
| 41 | SVEP1 | MFAP4 | TIMP1 | PDPN | NEGR1 | IGF1 | FNDC1 |
| 42 | THBS2 | TSHZ2 | MFAP4 | PLXDC2 | MOXD1 | F2R | NTM |
| 43 | TMEM119 | COL8A1 | CLEC11A | OMD | C1R | F3 | DPYSL3 |
| 44 | IGFBP3 | STEAP1 | INHBA | MMP23B | FGF7 | MMP2 | SLC6A6 |
| 45 | C1S | PTGDS | MXRA8 | LXN | COL1A1 | SFRP4 | CYP1B1 |

| Rank | T18 | T7 | T6 | T4 | T24 | T12 |
|---|---|---|---|---|---|---|
| 1 | DCN | CYP1B1 | COL10A1 | COL10A1 | DCN | MMP2 |
| 2 | SERP2 | SFRP2 | PDGFRA | SFRP2 | LUM | LUM |
| 3 | LUM | COL8A1 | SFRP2 | COL1A1 | FBLN1 | PDGFRA |
| 4 | C3 | PDGFRA | CYP1B1 | MMP2 | VCAN | CTHRC1 |
| 5 | MMP2 | COL10A1 | MMP2 | LUM | SFRP4 | ITGBL1 |
| 6 | APOD | SFRP4 | VCAN | COL1A2 | COL1A2 | EFEMP1 |
| 7 | EFEMP1 | CTHRC1 | CTHRC1 | CTHRC1 | MMP2 | SFRP2 |
| 8 | MFAP4 | APOD | LUM | DCN | SFRP2 | FBLN5 |
| 9 | FBLN1 | MMP2 | SFRP4 | CTSK | C1R | VCAN |
| 10 | SERP4 | PLXDC2 | APOD | MFAP2 | C1S | APOD |
| 11 | CCDC80 | VCAN | PLXDC2 | APOD | APOD | COL8A1 |
| 12 | RARRES1 | BNC2 | COL8A1 | MATN3 | CTSK | STEAP1 |
| 13 | C1S | MRC2 | FBLN1 | MEG3 | CCDC80 | COL1A2 |
| 14 | VCAN | DPYSL3 | OMD | ISLR | ISLR | PTGDS |
| 15 | PTGDS | COL1A2 | THBS2 | FBLN1 | C3 | ISLR |
| 16 | C1R | FBLN5 | FAP | COL11A1 | EFEMP1 | LXN |
| 17 | CTSK | CREB3L1 | MFAP2 | COL3A1 | MGP | OLFML3 |
| 18 | PDGFRA | COL3A1 | COL6A3 | SFRP4 | TSHZ2 | THBS2 |
| 19 | SERPINF1 | OMD | RARRES1 | CXCL14 | PDGFRA | FBLN1 |
| 20 | MOXD1 | RARRES1 | EFEMP1 | ITGBL1 | OMD | MGST1 |
| 21 | GPNMB | PODN | COL1A2 | COL6A3 | FBLN5 | MEG3 |
| 22 | RARRES2 | LSAMP | ANKH | VCAN | COL1A1 | PDPN |
| 23 | FBLN5 | THBS2 | FNDC1 | IGFL2 | OGN | MFAP2 |
| 24 | ISLR | SVEP1 | SPON2 | RARRES2 | SERPINF1 | DPT |
| 25 | ITGBL1 | ITGBL1 | PDPN | FNDC1 | CTGF | C3 |
| 26 | COL10A1 | PTGDS | CTSK | COL5A1 | FBLN2 | MRC2 |
| 27 | CYP1B1 | FAP | MFAP5 | OMD | COL6A3 | PTGIS |
| 28 | C7 | LUM | HTRA3 | CST4 | PODN | MOXD1 |
| 29 | PLXDC2 | FBLN1 | LRP1 | INHBA | LTBP2 | HSD11B1 |
| 30 | CTHRC1 | SULF1 | COL1A1 | GJB2 | ITGBL1 | SFRP4 |
| 31 | DPT | INHBA | DIO2 | IGFBP3 | C7 | LSAMP |
| 32 | CLU | SEMA3C | DCN | HTRA3 | PLXDC2 | PLXDC2 |
| 33 | NPC2 | LOX | ALDH1A3 | CST1 | MOXD1 | MFAP4 |
| 34 | MEG3 | PDPN | LOX | FBLN2 | TMEM119 | OGN |
| 35 | RP11- | IGF1 | FBLN2 | MFAP5 | COL8A1 | COL6A3 |
| | 572C15.6 | | | | | |
| 36 | MGP | LAMP5 | OLFML3 | MXRA5 | LRP1 | SPON2 |
| 37 | COL6A3 | FBLN2 | TMEM119 | THBS2 | RARRES1 | COL1A1 |
| 38 | LTBP2 | OGN | MEG3 | CTGF | BOC | LTBP2 |
| 39 | AEBP1 | PTGFRN | FGF7 | MRC2 | CYBRD1 | PODN |
| 40 | PDPN | GAS7 | TMSB10 | GJA1 | LXN | DCN |
| 41 | COL1A1 | CXCL14 | CXCL14 | FAP | LOX | CTSK |
| 42 | COL8A1 | COL11A1 | ITGBL1 | SPON2 | HTRA3 | SPOCK1 |
| 43 | S100A13 | FNDC1 | IGFBP3 | BICC1 | RP11- | COL3A1 |
| | | | | | 572C15.6 | |
| 44 | OGN | HTRA3 | CDH11 | EMP1 | CYP1B1 | RARRES1 |
| 45 | MMP23B | MOXD1 | IGF1 | FGF7 | CTHRC1 | SVEP1 |

| Rank | T1 | T5 | T22 | T11 | T21 | T23 | T9 | T16 | T17 | T8 |
|---|---|---|---|---|---|---|---|---|---|---|
| 1 | SFRP2 | PDGFRA | COL1A2 | LUM | COL10A1 | COL1A1 | LUM | LUM | COL10A1 | COL11A1 |
| 2 | VCAN | CYP1B1 | PDGFRA | DCN | CTHRC1 | COL1A2 | DCN | DCN | CTHRC1 | COL10A1 |
| 3 | LUM | SFRP2 | THBS2 | CTHRC1 | THBS2 | COL3A1 | RARRES2 | COL1A1 | COL11A1 | CREB3L1 |
| 4 | PDGFRA | SFRP4 | MMP2 | SFRP2 | GJB2 | COL6A3 | CTHRC1 | COL1A2 | ISLR | RP11- |
| | | | | | | | | | | 400N13.3 |
| 5 | COL1A2 | DPT | COL1A1 | COL10A1 | SFRP2 | LUM | SFRP2 | COL6A3 | MMP2 | SFRP2 |
| 6 | EFEMP1 | LUM | COL3A1 | RARRES2 | COL11A1 | FN1 | AEBP1 | COL3A1 | COL1A1 | BASP1 |
| 7 | DCN | MEG3 | ITGBL1 | AEBP1 | CCDC80 | COL5A2 | COL10A1 | VCAN | COL1A2 | PDPN |
| 8 | CCDC80 | EFEMP1 | COL10A1 | NBL1 | NBL1 | VCAN | NBL1 | SFRP2 | COL3A1 | BNC2 |
| 9 | ISLR | VCAN | CTHRC1 | CTSK | DCN | COL5A1 | MMP2 | MEG3 | AEBP1 | C5orf46 |
| 10 | SFRP4 | IGF1 | LUM | VCAN | AEBP1 | THBS2 | CTSK | CTHRC1 | MMP11 | PLXDC2 |
| 11 | COL6A3 | FBLN5 | SFRP2 | CTGF | INHBA | SFRP2 | THBS2 | EFEMP1 | THBS2 | SPOCK1 |
| 12 | CYP1B1 | SERPINE2 | EFEMP1 | COL8A1 | LUM | CTHRC1 | FBLN1 | PDGFRA | COL12A1 | ADM |
| 13 | COL1A1 | FBLN1 | MRC2 | THBS2 | FBLN1 | MMP2 | VCAN | FBLN2 | HTRA1 | MMP2 |
| 14 | APOD | SCN7A | COL6A3 | MMP2 | MMP2 | COL10A1 | CCDC80 | LOX | MMP14 | ARL4C |
| 15 | CLDN11 | LTBP2 | PDPN | COL11A1 | COL6A3 | COL11A1 | COL1A1 | COL5A1 | SFRP2 | MEG3 |
| 16 | CTSK | APOD | VCAN | INHBA | OMD | AEBP1 | S100A6 | COL8A1 | SULF1 | GJA1 |
| 17 | FBLN1 | PTGDS | DPYSL3 | C1QTNF3 | MEG3 | COL12A1 | COL8A1 | LXN | LUM | VCAN |
| 18 | MMP2 | ISLR | LOX | SFRP4 | COL8A1 | DCN | TMSB10 | MMP2 | SDC1 | FIBIN |
| 19 | COL3A1 | PODN | GJA1 | MATN3 | ISLR | SPARC | C1S | S100A10 | DCN | COL1A2 |
| 20 | PTGDS | DCN | APOD | HTRA1 | MMP11 | TMSB10 | COL1A2 | FBLN1 | MFAP5 | ZFHX4 |
| 21 | FBLN2 | MMP2 | FAP | ITGBL1 | MFAP5 | MMP14 | COL3A1 | ISLR | VCAN | MFAP2 |
| 22 | CTHRC1 | MGP | MXRA5 | MFAP5 | PPAPDC1A | SDC1 | HTRA1 | FAP | COL6A3 | MME |
| 23 | C3 | C7 | PODN | CXCL14 | CTSK | POSTN | CD99 | PPIC | GJB2 | MFAP5 |
| 24 | PLXDC2 | MGST1 | CXCL14 | GJB2 | MXRA5 | FBLN1 | ISLR | CYP1B1 | GREM1 | RAB3B |
| 25 | RARRES1 | SPOCK1 | COL8A1 | IGFBP3 | FNDC1 | INHBA | SERPINF1 | CREB3L1 | TIMP2 | ITGBL1 |
| 26 | RP11-572C15.6 | CCDC80 | SFRP4 | CCDC80 | COL1A1 | SERPINH1 | TSC22D3 | CCDC80 | COL5A2 | GJB2 |
| 27 | THBS2 | SLC19A2 | SEMA3C | CD99 | SDC1 | MXRA5 | FTL | MRC2 | FAP | COL3A1 |
| 28 | C7 | HTRA3 | LRP1 | C1S | VCAN | HTRA1 | MFAP2 | MFAP5 | MFAP2 | NTM |
| 29 | LAMA2 | MOXD1 | NTM | LOXL1 | GREM1 | MMP11 | ANXA2 | THBS2 | CTSK | PDLIM4 |
| 30 | OLFML3 | ITGBL1 | C3 | FIBIN | PDPN | ISLR | LAPTM4A | CTSK | COL5A1 | CMTM8 |
| 31 | MFAP4 | CTSK | COL11A1 | FBLN1 | FBLN2 | MEG3 | NNMT | MXRA5 | PPAPDC1A | TANC2 |
| 32 | SLIT2 | FBLN2 | UNC5B | PALLD | MFAP2 | TIMP2 | C1R | OGN | FN1 | NT5E |
| 33 | IGF1 | CTHRC1 | LOXL1 | SDC1 | C1QTNF3 | FSTL1 | RPL27A | RARRES2 | TMEM158 | TENM3 |
| 34 | LRP1 | SVEP1 | COL5A2 | MFAP2 | COL5A2 | COL6A2 | INHBA | FSTL1 | POSTN | EPDR1 |
| 35 | PDLIM3 | MXRA5 | SCARA3 | ANXA2 | CDH11 | CTSK | NUPR1 | COL5A2 | ANTXR1 | MYH10 |
| 36 | LTBP2 | STEAP1 | COL8A2 | APOD | LOX | MFAP2 | LGALS1 | LRP1 | PLAU | LOX |
| 37 | FBLN5 | NEGR1 | PTGDS | OMD | COL3A1 | MFAP5 | COL6A3 | RARRES1 | INHBA | COL8A1 |
| 38 | MGP | C3 | CCDC80 | ISLR | PDGFRA | GAS1 | COL11A1 | FBLN5 | GJA1 | EVA1A |
| 39 | MEG3 | RARRES1 | ALDH1A3 | SLC6A6 | F13A1 | LRRC15 | OMD | FBN1 | LGALS1 | MXRA5 |
| 40 | BICC1 | RP11- | FGFR1 | CYR61 | FRMD6 | COL8A2 | CD55 | GAS1 | PTK7 | BICC1 |
| | | 572C15.6 | | | | | | | | |
| 41 | MRC2 | PLXDC2 | BOC | COL1A2 | APOD | FBLN2 | PDPN | BNC2 | CD99 | C1orf198 |
| 42 | FGFR1 | LRP1 | PLXDC2 | MMP11 | COL1A2 | GREM1 | LOXL1 | PLOD2 | NBL1 | INHBA |
| 43 | ABL2 | ABI3BP | MFAP2 | MEG3 | DIO2 | APOD | FIBIN | PDPN | NTM | PDGFC |
| 44 | MXRA5 | FAP | MFAP5 | PLXDC2 | CD55 | COL8A1 | NTM | MMP23B | RARRES2 | FBLN2 |
| 45 | RGS2 | COL6A3 | TMSB10 | FBLN2 | HTRA1 | CD99 | S100A10 | LTBP2 | FBLN1 | B4GALT1 |

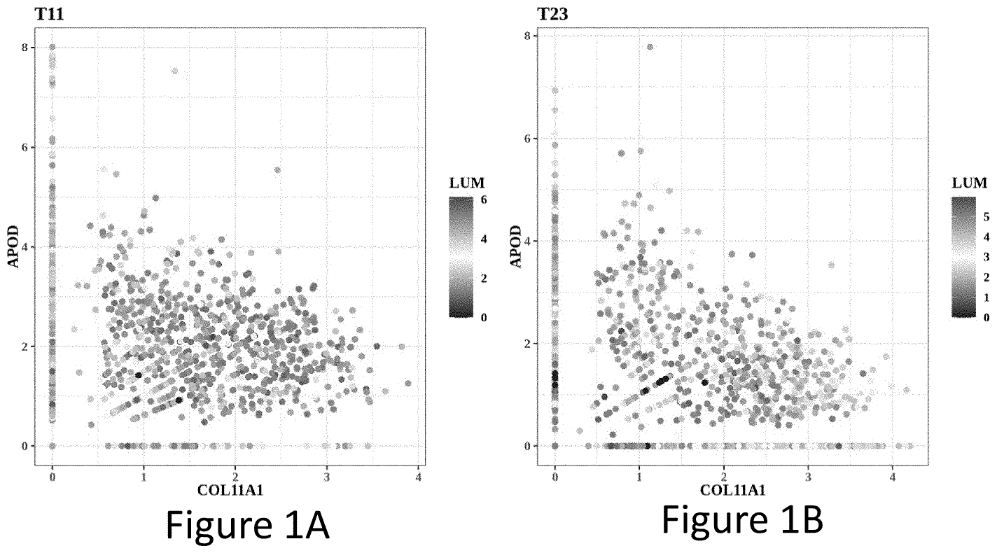

Further demonstration of the continuity of the transition: As an additional confirmation of the continuity of the transition (as opposed to the presence of a mixture of distinct fibroblastic subtypes), FIG. 1 shows scatter plots for genes APOD and COL11A1, color-coded for the expression of fibroblastic marker LUM, of the mesenchymal cells in two fibroblast-rich samples T11 and T23. The presence of cells covering the full range from the upper-left to the bottom-right sides of the plots, including the intermediate stages in which cells co-express both markers, demonstrates the presence in each sample of cells representing the continuously varying transition from ASCs to COL11A1-expressing CAFs.

To further investigate the continuous transition, the 34 pancreatic samples were partitioned into three groups. Group 1 includes the eleven normal samples (N1 to N11). For tumor samples, the rearranged samples in Table 5 were divided into two groups (Group 2 and Group 3). Group 2 contains all samples to the left of and including T22, so that APOD is ranked before COL11A1 in the attractors of that Group, representing a relatively earlier stage of this transition. Then, the consensus version of the attractor finding algorithm was applied, and the signatures representing the main state of the fibroblasts for each of the above three sample groups (Table 6) were identified. Although there are many shared genes, the groups have distinct gene rankings. Group 1 (normal samples) contains many adipose-related genes, consistent with Table 2. Group 3 contains, in addition to COL11A1, many among the other CAF genes, such as THBS2, INHBA, AEBP1, MFAP5 and COL10A1. Group 2 displays an intermediate state, including markers of both ASCs as well as CAFs.

TABLE 6
Top 30 genes of the consensus attractors for three different
PDAC sample groups. Group1: normal samples; Group3: T11,
T21, T23, T9, T16, T17, T8; Group2: other tumor samples.

| Rank | Group1 | Group2 | Group3 |
|---|---|---|---|
| 1 | LUM | LUM | COL1A1 |
| 2 | DCN | SFRP2 | COL1A2 |
| 3 | FBLN1 | APOD | COL3A1 |
| 4 | C7 | SFRP4 | FN1 |
| 5 | APOD | MMP2 | COL5A2 |
| 6 | PTGDS | VCAN | COL5A1 |
| 7 | SFRP2 | PDGFRA | COL6A3 |
| 8 | C1S | FBLN1 | COL11A1 |
| 9 | CCDC80 | DCN | CTHRC1 |
| 10 | MGP | EFEMP1 | THBS2 |
| 11 | DPT | CTHRC1 | VCAN |
| 12 | CXCL12 | ISLR | COL10A1 |
| 13 | C1R | COL6A3 | LUM |
| 14 | FBLN5 | COL1A2 | SPARC |
| 15 | C3 | CTSK | COL12A1 |
| 16 | PDGFRA | CYP1B1 | MMP2 |
| 17 | SRPX | FBLN5 | DCN |
| 18 | COL6A3 | MEG3 | SFRP2 |
| 19 | ADH1B | COL1A1 | TMSB10 |
| 20 | CFD | C3 | POSTN |
| 21 | OLFML3 | RARRES1 | MXRA5 |
| 22 | SERPINF1 | CCDC80 | COL6A2 |
| 23 | MMP2 | MOXD1 | ISLR |
| 24 | CST3 | PLXDC2 | AEBP1 |
| 25 | SEPP1 | HTRA3 | MEG3 |
| 26 | ABCA8 | COL10A1 | MFAP5 |
| 27 | COL1A2 | COL8A1 | SERPINH1 |
| 28 | LAMB1 | ITGBL1 | MMP14 |
| 29 | SVEP1 | OMD | MFAP2 |
| 30 | MEG3 | PTGDS | INHBA |

To find potential critical genes at the initiation phase of the cellular transition, the first tumor samples (with the highest APOD ranking) in Table 5 were compared with those of the normal ASCs.

Gene SFRP4 stands out, as it appears for the first time remarkably among the top genes in all the first samples T2, T13, T14, T19, ranked 4th, 6th, 4th 8th, respectively. This suggests that the Wnt pathway is involved in the initiation of the cellular transition because SFRP4 is a Wnt pathway regulator whose expression has been found associated with various cancer types [25,26]. Interestingly, SFRP4 disappears from the list of the attractors, indicating that it is downregulated in the final stage of the transition.

It is also known that gene RARRES1 (aka TIG1) plays an important role in regulating the proliferation and differentiation of ASCs [27]. Consistently, Table 6 reveals that RARRES1 appears for the first time in the attractors of the initial tumor samples. Just like SFRP4, RARRES1 is downregulated in the final stage, related to the fact that it has been suggested as a tumor suppressor [28,29].

Differential expression (DE) analyses were performed comparing the normal samples with the first samples (T2, T13, T14, T19) of Table 5. The results of such DE analysis represent the full population of fibroblasts and do not necessarily reflect the expression changes in the particular cells undergoing the ASC to COL11A1 expressing CAF transition. Gene CFD was found to be most downregulated, consistent with the expected downregulation of adipose-related genes as they differentiate into fibroblasts. Genes SFRP4 and RARRES1 are upregulated, consistent with their appearance in the attractors.

On the other hand, the top upregulated gene is phospholipase A2 group IIA (PLA2G2A), which is not among the top genes of any identified attractors, indicating that it is not expressed by cells undergoing the ASC to COL11A1-expressing CAF transition. It probably still plays, however, an important related parallel role and many previous studies referred to its effects on the prognosis of multiple cancer types [30-32]. The PLA2G2A protein is a member of a family of enzymes catalyzing the hydrolysis of phospholipids into free fatty acids. This process can lead to fatty acid oxidation, which may facilitate metastatic progression. Indeed, it has been recognized that fatty acid oxidation is associated with the final COL11A1-expressing stage of the transition [33]. These results suggest that lipid metabolic reprogramming plays an important role in the metastasis-associated biological mechanism [34] by potentially providing energy for the metastasizing tumor cells.

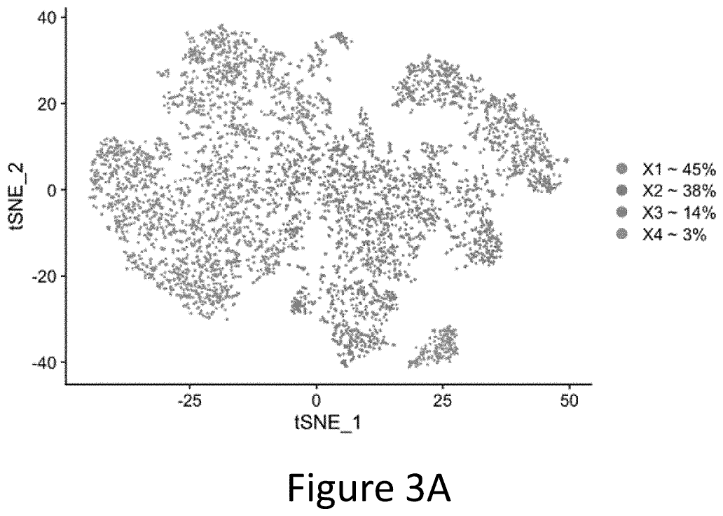

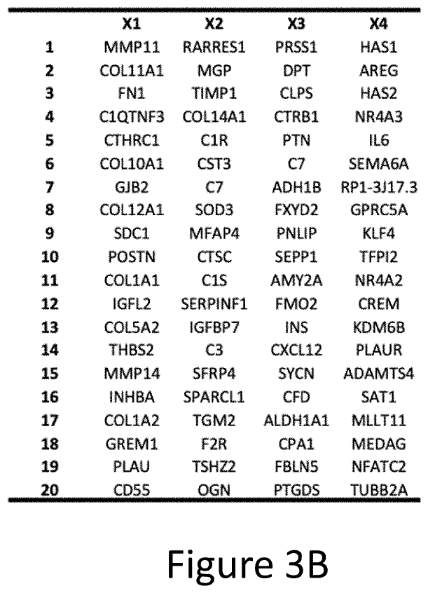

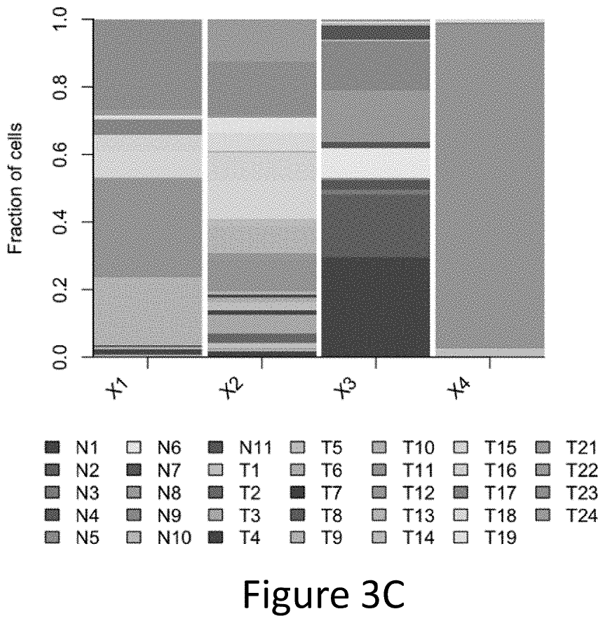

Validation with trajectory inference: Trajectory inference (TI) analysis was independently applied on the PDAC fibroblasts by using the Slingshot [35] method in an unsupervised manner. Unsupervised clustering was first performed on the identified fibroblasts, resulting in four subgroups X1, X2, X3, X4 (FIG. 3A) with the top differentially expressed genes shown in FIG. 3B. One of these clusters (X4) was discarded from further TI analysis because it mainly expressed the IL1 CAF marker HAS1 (Hyaluronan Synthase 1), which is not expressed by either ASCs or COL11A1-expressing CAFs, and contained only 3% of fibroblasts resulting almost exclusively from patient T11 (FIG. 3C).

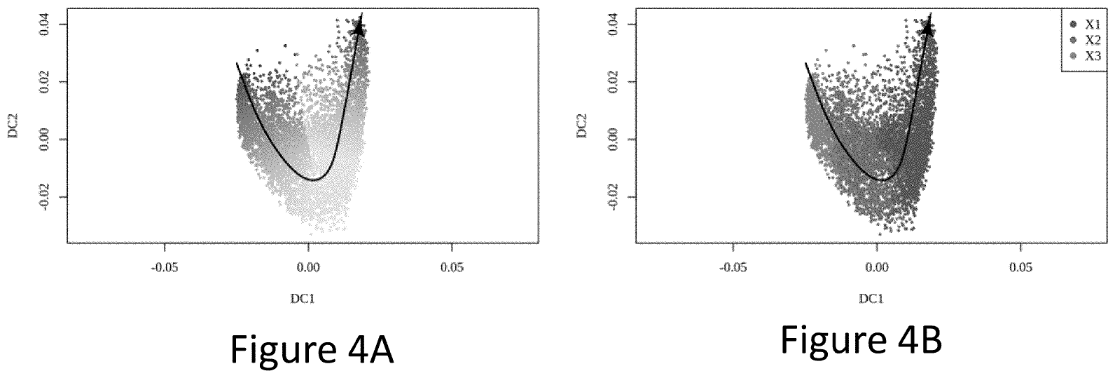

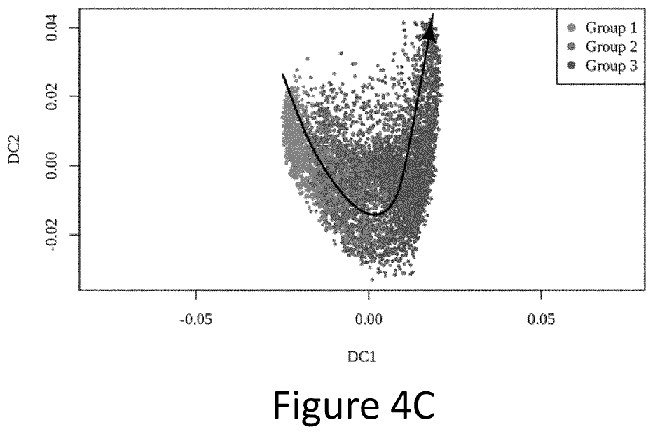

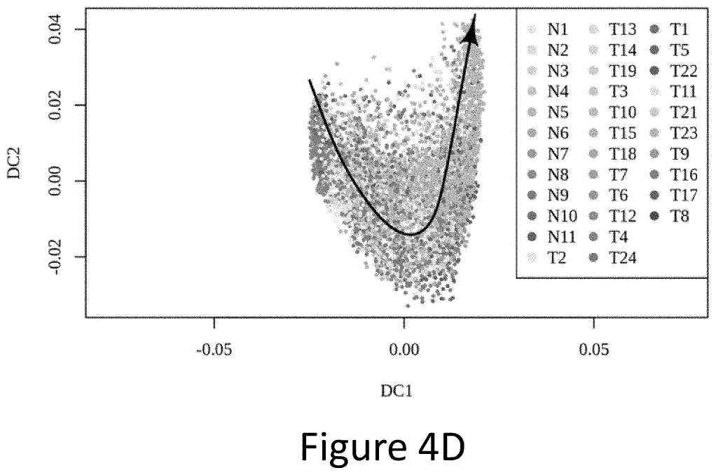

As seen from the list of top differentially expressed genes of each cluster, X1 contains CAF genes top-ranked (including MMP11, COL11A1, THBS2, INHBA), X2 has RARRES1 at the top, and X3 has ASC genes top-ranked, including DPT, C7, CXCL12 and CFD. Consistently, FIGS. 4A and 4B show the single trajectory path resulting from TI analysis, where X3 is the starting point, and X1 is the endpoint of the trajectory, while X2 (highly expressing RARRES1) is an intermediate point, thus validating the continuous ASC to COL11A1-expressing CAF transition. The orderings of patient groups and sample identity (FIGS. 4C and 4D) are also consistent with the findings based on attractor analysis. The following top 100 genes have zero P-value, are ranked by their variances, and are resulted from pseudotime-based differential gene expression analysis: APOD, COL1A1, PTGDS, C7, MGP, CXCL14, CTGF, IGFBP5, FN1, MMP11, ACTA2, CFD, SFRP4, INS, COL3A1, CTHRC1, IGFBP3, TAGLN, MT1X, MT2A, POSTN, TIMP1, C3, FOS, COL1A2, COL10A1, ZFP36, GSN, TIMP3, SPARC, DUSP1, CST1, APOE, JUNB, SPARCL1, CCDC80, ASPN, FBLN1, COL11A1, COL5A2, SERPINE1, IFI27, THBS2, VCAN, S100A4, MFAP4, DPT, ID3, AEBP1, SEPP1, TPM1, COL6A3, DDIT4, COMP, MMP14, SERPINF1, C1QTNF3, MMP2, COL12A1, INHBA, HOPX, SPINK1, PRSS23, MT1E, MFAP5, CTSC, COL5A1, RARRES1, SOD3, IGFBP4, MYL9, TPM2, PLA2G2A, A2M, S100A10, BGN, C10orf10, COL6A1, CTSK, NBL1, C1R, TXNIP, TSC22D1, CYP1B1, HTRA1, CLU, LUM, NNMT, RPS4Y1, ALDH1A1, CXCL12, PRSS1, GADD45B, RARRES2, IGFBP7, EFEMP1, IFI6, PRELP, SERPINH1, and COL8A1. Top-ranked several ASC genes were identified, as well as CAF genes, while some general fibroblastic markers, such as DCN, are missing, consistent with the continuity of the ASC to COL11A1-expressing CAF transition. A generalized additive model (GAM) was used to fit to pseudotime-ordered expression data to visualize the trend of gene expressions (FIG. 2A).

There was a prominent difference between adipose-related genes and COL11A1-associated genes. The expression of the adipose-related genes steadily fell across the process (FIG. 2B), while the expression of COL11A1-associated genes gradually increased (FIG. 2C). There is a significant negative correlation between these two groups of genes, e.g., COL11A1 (the last among those genes to increase its expression) was exclusively overexpressed in the mature CAFs, which did not express C7. Of particular interest, genes SFRP4 and RARRES1 (FIG. 2D) increased consistently at the beginning and then decreased after reaching a peak, suggesting that they may play important roles in the differentiation path.

Validation in other cancer types: Next, the ASC to COL11A1-expressing CAF transition in other solid cancer types was validated. Those containing a large (at least 100) number of fibroblasts were selected and separately analyzed each of them, obtaining consistent results. Specifically, four scRNA-seq datasets from head and neck cancer (HNSCC) [36], ovarian cancer[37], lung cancer [38] and breast cancer [39] were used.

The COL11A1-expressing CAF signature has been confirmed to be a pan-cancer signature [40-42]. Therefore, the most important validation task would be to confirm the existence of the APOD/CFD/CXCL12/MGP/PTGDS-expressing ASCs as the starting point of the transition and to also confirm that some samples are at an intermediate stage, expressing genes such as SFRP4, RARRES1 and THBS2, in addition to the core ASC genes, demonstrating that they are at an intermediate stage of the transition.

Head and neck squamous cell carcinoma: For the HNSCC dataset, the authors of the paper presenting the data [36] reported that the cancer-associated fibroblasts in the dataset can be partitioned into two subsets, which they name CAF1 and CAF2. The disclosed results show the top three differentially expressed genes of the CAF2 group are CFD, APOD and CXCL12, while the full gene list for CAF2 also includes genes MGP, C3, C7, DPT, PTGDS. This strongly suggests that the partitioning used in the paper was influenced by the presence of an ASC cell subpopulation, identical, or at least very similar to, those discovered in the PDAC. Similarly, the list of differentially expressed genes for CAF1 includes genes INHBA, THBS2, CTHRC1, POSTN, MMP11, COL5A2, COL12A1, suggesting that the identified CAF1 subpopulation was influenced by the presence of differentiated CAFs, which would eventually express COL11A1. Finally, gene RARRES1 also appears among the list of CAF2 genes, suggesting that it was captured among cells that had started the process of ASC to COL11A1-expressing CAF transition.

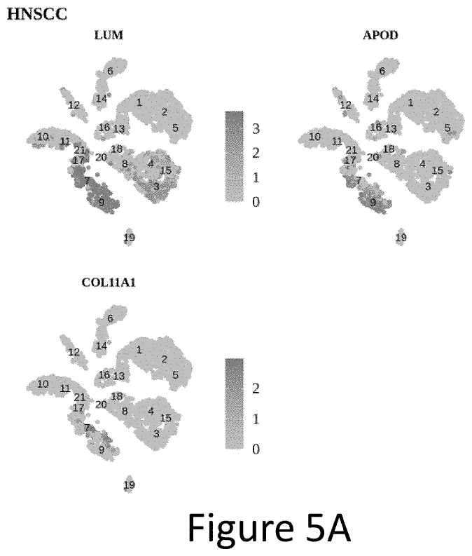

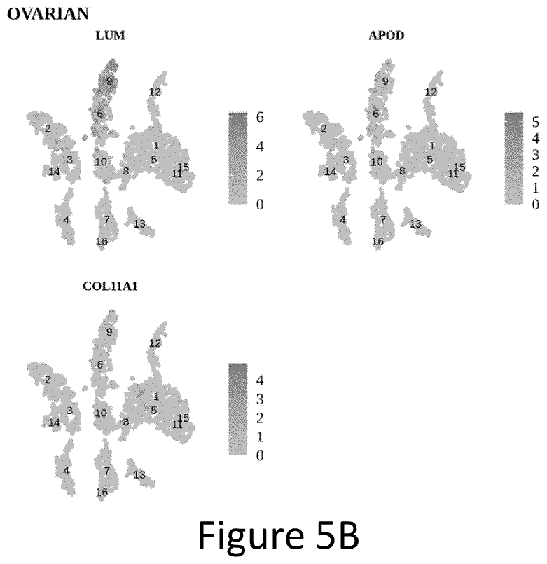

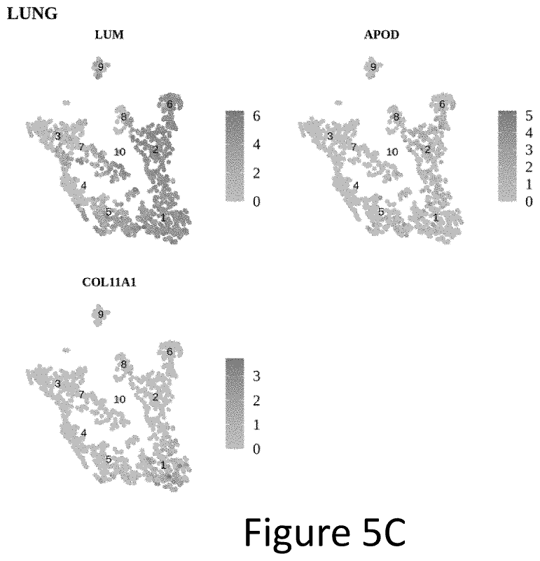

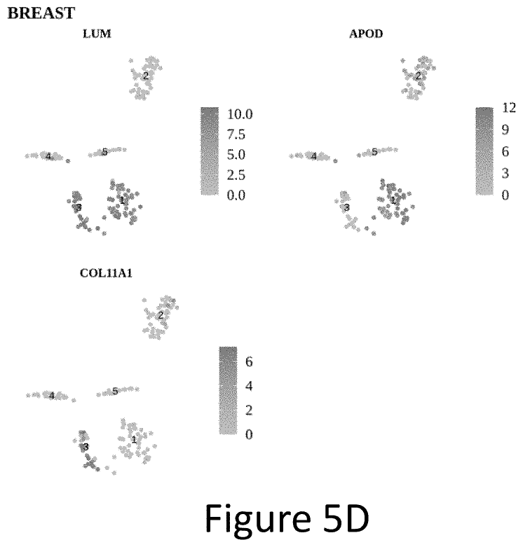

In the independent analysis, clustering was performed, identifying 1,026 fibroblasts from all available cells (FIG. 5A). There were two fibroblastic clusters (X7 and X9) expressing CAF associated genes (COL11A1, COL12A1, MMP11, INHBA, THBS2, COL10A1, COL8A1, FN1) and ASC associated genes (APOD, C7, PTGDS), respectively, which confirmed the presence of these two populations in HNSCC.

Among the individual patients, the most prominent case is sample HNSCC28, which contains a rich set of cells undergoing differentiation. Applying the attractor finding algorithm on the fibroblasts of that sample resulted in genes LUM, APOD, COL6A3, PDGFRA, DCN, and CFD being among the top-ranked, revealing that it represents an ASC population. Furthermore, the presence of genes THBS2, MFAP5 and VCAN in the same attractor reveals that these cells have already started undergoing the transition.

Ovarian cancer: For the ovarian dataset, the clustering results showed two clusters (X6 and X9) expressing COL11A1-associated genes and ASC-associated genes, respectively (FIG. 5B). Among the individual patients, the most validating gene was HG2F and LG2, both of whose datasets consistently contain cells from the fatty omental tissue. The result includes the corresponding two attractors identified in the cells of each patient. Among the top-ranked genes for HG2F are DCN, LUM, C1S, C7, and C3, but also RARRES1, suggesting that they represent fibroblasts undergoing the transition, while the LG2-based attractor contains highly ranked all three genes COL11A1, INHBA, THBS2.

Lung cancer: The dataset contains a large number (>50,000) of cells but was only classified −2% (=1,346) among them as mesenchymal cells, including fibroblasts and pericytes (Materials and Methods). Among those cells, there were two fibroblastic clusters (X1 and X2) expressing related genes (COL11A1, COL12A1, MMP11, INHBA, THBS2, COL10A1, COL8A1, FN1) and ASC related genes (APOD, C7, PTGDS), respectively (FIG. 5C). The presence of the transition is evident by the attractors identified in the mesenchymal cells for patients 4 and 3. The former prominently contains genes CFD, PTGDS and C7, while the latter contains THBS2, COL10A1 and INHBA.

Breast cancer: The size of the breast cancer dataset is small (−1,500 cells in total), and 169 cells among them were classified as mesenchymal (Materials and Methods). By further clustering, these cells, ASCs (X1) and COL11A1-expressing CAFs (X3), were identified (FIG. 5D). ASC-related genes (APOD, MFAP4, CFD) were identified in X1, while CAF-related genes (COL10A1, COL11A1, MMP11, INHBA, FN1, THBS2, AEBP1, COL12A1) are among the top 15 of X3. Patients PT089 and PT039 contain the highest proportions (>50%) of the ASC and COL11A1-expressing CAF subpopulations, respectively, and consistent results in their attractors were observed, as the former contains C1S, C1R, CXCL12, PTGDS, C3, while the latter contains THBS2, COL11A1, COL10A1, at top-ranked positions.

Potential therapeutic targets inhibiting the invasiveness-associated transition: This work provides opportunities for identifying therapeutic targets inhibiting the cellular transition. For example, targeting of gene MFAP5 was recently found to enhance chemosensitivity in ovarian and pancreatic cancers [43]. Specifically, the author states that “MFAP5 blockade suppresses fibrosis through downregulating of fibrosis-related genes such as COL11A1.” Consistently, MFAP5 was one of the most highly associated genes with COL11A1 (Table 4).

As mentioned earlier, genes SFRP4 and RARRES1 are transiently expressed in Group 2 of Table 6, suggesting that they can be investigated for inhibiting the cellular transition. Of particular interest as potential drivers are noncoding RNAs due to their typical regulatory role. Because the expression of these genes is not accurately captured by scRNA-seq technology, a thorough analysis of the full set of The Cancer Genome Atlas (TCGA) pan-cancer data was performed. For the RNA sequencing and miRNA sequencing dataset of each cancer type, the genes in which more than 50% of the samples have zero counts were removed. Then quantile normalization was performed using the limma package [44] (v3.40.6) on log 2 transformed counts. In each of the 33 cancer types, all protein-coding genes were ranked in terms of the association (using the metric of mutual information) of their expression with that of gene COL11A1. The 11 cancer types (LGG, SKCM, SARC, LAML, PCPG, GBM, TGCT, THYM, ACC, UVM, UCS), in which neither THBS2 nor INHBA was among the 50 top-ranked genes, were excluded because of the absence of significant amounts of COL11A1-expressing CAFs in those samples. In each of the remaining 22 cancer types, then all long noncoding RNAs (lncRNAs) and microRNAs (miRNAs) were ranked in terms of their association with COL11A1. Finally, pan-cancer sorting of all lncRNAs and miRNAs was performed in terms of the median rank of all lncRNAs and miRNAs.

LINC01614 represents a particularly promising therapeutic target. It had a perfect score of 1 in the pan-cancer sorting list, being strikingly the top-ranked gene in 14 (BRCA, UCEC, KIRC, HNSC, LUAD, LUAD, LUSC, OV, STAD, ESCA, PAAD, MESO, DLBC, CHOL) out of the 22 cancer types. In fact, the association of LINC01614 was even higher than that of marker protein-coding gene INHBA. The pan-cancer consensus ranking of protein-coding genes in terms of LINC01614 corresponds precisely to the COL11A1-expressing CAF signature. These rankings, in which marker genes unique to the original and intermediate stages are missing, indicate that LINC01614 is involved in the very final stage of the creation of the COL11A1-expressing CAFs. Therefore, therapeutics targeting LINC01614 specifically in patients' CAFs may inhibit the final metastasis-facilitating stage of the transition. The three top-ranked miRNAs were miR-199a-1, miR-199b, miR-199a-2. The associated miR-214 is also very highly ranked.

The results indicate that cancer invasiveness-associated COL11A1-expressing CAFs are produced as a result of the interaction of tumor cells with the adipose microenvironment. Therefore, the disclosed subject matter provides an explanation for the fact that adipose tissue contributes to the development and progression of cancer [45-47].

The disclosed subject matter provides methods for precisely identifying the ASC population. The marker genes among the top-ranked attractor genes are shown in each of the eleven columns of Table 2. The identification of those particular marker genes (APOD prominent among them) cannot be due to chance because these were eleven totally independent unbiased experiments, and also because the attractor algorithm applied on the SVF of normal adipose tissue in another independent dataset identified precisely the same genes. This finding could not have been achieved with traditional methods.

There is consensus agreement that CAFs are a promising potential target for optimizing therapeutic strategies against cancer, but such developments are restricted by our current limitations in our understanding of the origin of CAFs and heterogeneity in CAF function [48]. Therefore, there is an urgent need to enhance our understanding of those matters. The disclosed results provide clarity on one important particular component (out of several) of the heterogeneous fibroblast tumor microenvironment. To avoid potential erroneous conclusions after applying bioinformatics algorithms, single-cell data analysis provides an unprecedented capability to validate results, including those resulting from the attractor algorithm, by “seeing” individual cells in color-coded scatter plots, such as the one shown in FIG. 1, observing and confirming the presence or absence of distinct populations characterized by the combined presence of particular marker genes.

In particular, there are several published papers relying on the application of clustering algorithms following dimensionality reduction on the particular datasets they use and concluding that there exist a number of distinct and mutually exclusive CAF subpopulations. These reported fibroblastic subpopulations occasionally have gene expression profiles that conflict with each other in significant ways among these publications. Examples include the hC1 and hC0 clusters in [49], the C9 and C10 clusters in [42], the CAF2 and CAF1 clusters in [36], the iCAF and myCAF clusters in [50,51] and the iCAF an mCAF clusters in [52]. A review of such results in pancreatic cancer appears in [53].

As an example of conflicting results, the “iCAFs” identified in [52] have significant differences from those identified in other papers and are, in fact, identical to the normal ASCs (FIG. 3B of [52]) identified in this paper, as evidenced by the list of its differentially expressed genes (PTGDS, LUM, CFD, FBLN1, APOD, DCN, CXCL14, SFRP2, MMP2, all of which appear in Table 3, further validating the ASC signature. Therefore, this identified cluster contains mainly normal cells at the origin of the transition, which should not even be called CAFs.

Similarly, a recent single-cell data analysis [54] identified two clusters “touching” each other in a UMAP plot (FIG. 2A of [54]), C0 and C3, which are precisely the two endpoints of the ASC to COL11A1-expressing CAF transition. Indeed, as identified in Table S6-1 of [54], C0 cluster has the marker genes APOD, PTGDS, C7, C3, MGP, which the attractor algorithm had identified and validated in this paper. On the other hand, the marker genes of cluster C3 are precisely those of the COL11A1-expressing CAFs, in which all three genes COL11A1, INHBA and THBS2 are top-ranked (because the metastatic process was already underway). Importantly, as shown in FIG. 2B of [54], the ASC marker genes APOD and PTGDS (top-ranked in C0 and unrelated to CAFs) are significantly expressed even in the COL11A1-expressing cluster C3 of the paper, providing further evidence of the presence of intermediate states consistent with the transition—and the separating line between C0 and C3 in the diagram is not generated by any biologically reliable manner, consistent with the continuity.

On the other hand, the disclosed results are consistent with and complementary to the results of [49] focusing on the immunotherapy response, in which the presence of the “TGF-beta CAFs” was inferred by an 11-gene signature consisting of MMP11, COL11A1, C1QTNF3, CTHRC1, COL12A1, COL10A1, COL5A2, THBS2, AEBP1, LRRC15, ITGA11. This population apparently represents the COL11A1-expressing CAF endpoint of the transition, and gene LRRC15 was selected as the representative gene based on the fact that it was found to be the most differentially expressed gene between CAFs and normal tissue fibroblasts in mouse models. Indeed, LRRC15 is a key member of the COL11A1-expressing CAF signature (Table 4 of [1]), and COL11A1 is the highest associated gene to LRRC15 in the Group 3 PDAC patients.

A detailed gene association-based scrutiny of all our results, including numerous color-coded scatter plots, was used rather than blindly accepting clustering results. This nontraditional computational methodology, when used on rich single-cell data, represents a paradigm shift in which systems biology alone can be trusted, by itself, for producing reliable results.

### Example 2: Development of Therapeutics Inhibiting the Transition of ASCs to CAFs

To test the effect of cancer cells on ASC in direct co-culture and enable subsequent cell separation, the following human breast carcinoma cell lines were stably transduced with a vector expressing green fluorescent protein (GFP): SUM149, MDA-MB-231 and MCF7. Human immortalized ASC (imASC) from subcutaneous fat, ASC from subcutaneous AT (scASC) from a healthy donor, and mammary fat ACS (maASC) from a healthy donor were stably transduced with a lentivirus pLVX-tdTomato-C1 (Addgene) expressing a red fluorescent protein, tdTomato red fluorescent protein (RFP) and carrying Puromycin selection gene. Gene mRNA expression was measured by RT-PCR.

In all experiments, for gene expression analysis, total RNA was extracted using the Trizol Reagent (Life Technologies, 15596018). Complementary DNAs were generated using High-Capacity cDNA Reverse Transcription Kit (Applied Biosystems, 4368814). PCR reactions were performed on a CFX96 Real-Time System C1000 Touch thermal cycler (Bio-Rad) using Q-PCR Master Mix (Gendepot, Q5600-005). Expression of mouse Phb, Ucp1, and Cox IV was normalized to 18S RNA. The Sybr green primers were as follows: LINC01614 Forward TCAACCAAGAGCGAAGCCAA (SEQ ID NO: 1), Reverse TTGGACACAGACCCTAGCAC (SEQ ID NO: 2); COL11A1 Forward TGGTGAT CAGAATCAGAAGTTCG (SEQ ID NO: 3), Reverse AGGAGAGTTGAGAATTGGGAATC (SEQ ID NO: 4); THBS2 Forward CAGTCTGAGCAAGTGTGACACC (SEQ ID NO: 5), Reverse TTGCAGAGACGGATGCGTGTGA (SEQ ID NO: 6); SFRP4 Forward CTATGACCGTGGCGTGTGCATT (SEQ ID NO: 7), Reverse GCTTAGGCGTTTACAGTCAACATC (SEQ ID NO: 8); 18S RNA Forward AAGTCCCTGCCCTTTGTACACA (SEQ ID NO: 9), Reverse GATCCGAGGGCCTCACTAAAC (SEQ ID NO: 10).

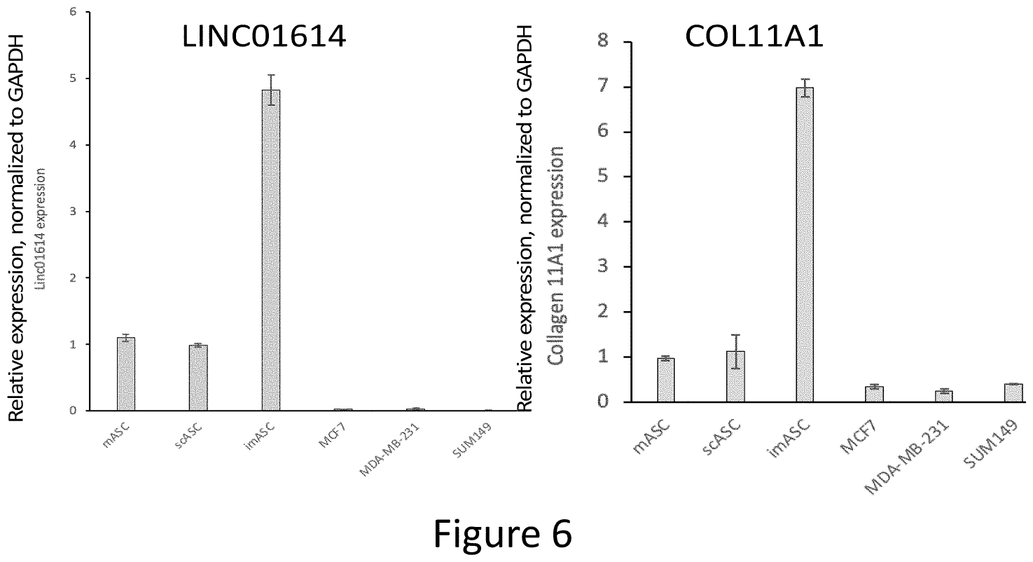

Cancer cells were found to express only the background level of LINC01614 (LNC01614) and COL11A1, the marker of aCAF. Both LINC01614 and COL11A1 were found to be expressed higher in imASC than in freshly-isolated scASC and mASC, likely due to prolonged culture passaging (FIG. 6).

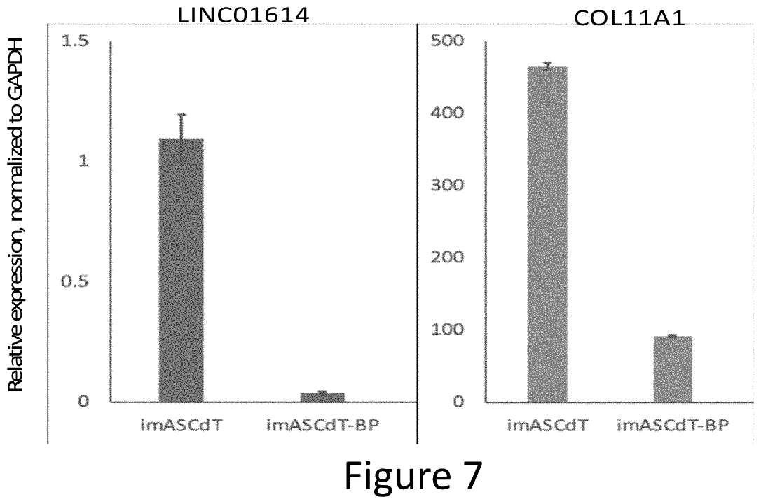

The red-fluoresence ASC were subsequently transduced with a lentivirus Lenti-CRISPR v2-Blast (Addgene #83480) carrying Blasticidin selection gene and guide RNA for LINC01614 CRISPR knock-out (1(0). This sgLINC01614 sequence is CACCGGTGTAAGGTACTCAAGTGCT (SEQ ID NO: 11). Upon Blasticidin/Puromycin (BP) selection, the LINC01614 KO cells were confirmed to lack LINC01614 expression, as measured by RT-PCR. Importantly, LINC01614-KO ASC had dramatically reduced COL11A1 expression, compared to control imASC expressing dTomato only (dT) (FIG. 7). This indicates that LINC01614 controls the expression of COL11A1.

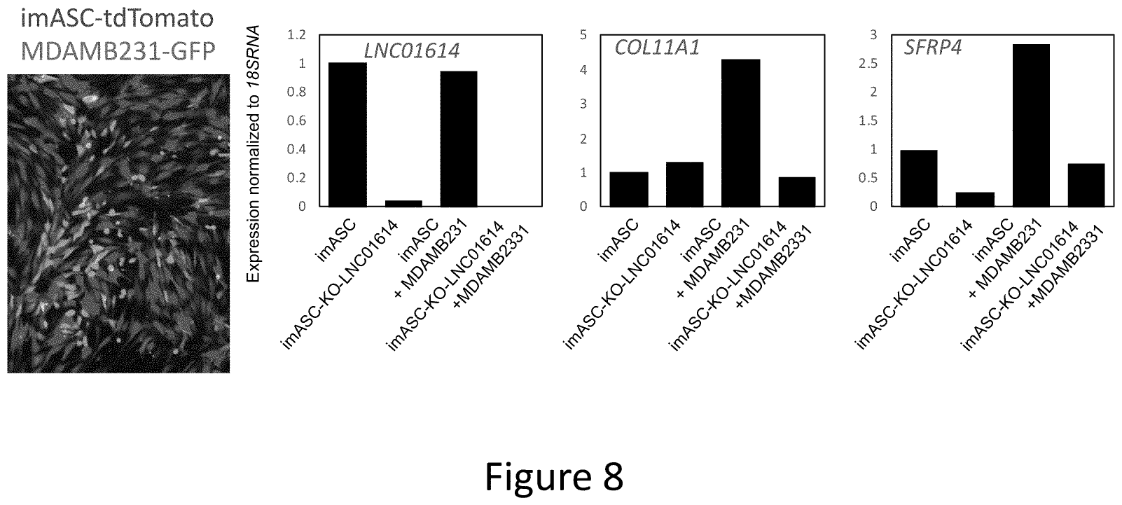

Changes in imASC induced by co-culture with MDAMB231 cancer cell, the aggressive cell line, in which the epithelial-mesenchymal transition (EMT) has occurred, were measured. Upon co-culture trypsin digestion, RFP+ cells were separated from co-cultures by fluorescence-activated cell sorting (FACS) using FACS-ARIA II, and gene expression analysis was performed. COL11A1 and SFRP4 expression was induced in imASC by MDAMB231 co-culture (FIG. 3). In contrast, COL11A1 and SFRP4 expression was not induced in LINC01614-KO imASC co-cultured with MDAMB231 cells (FIG. 8).

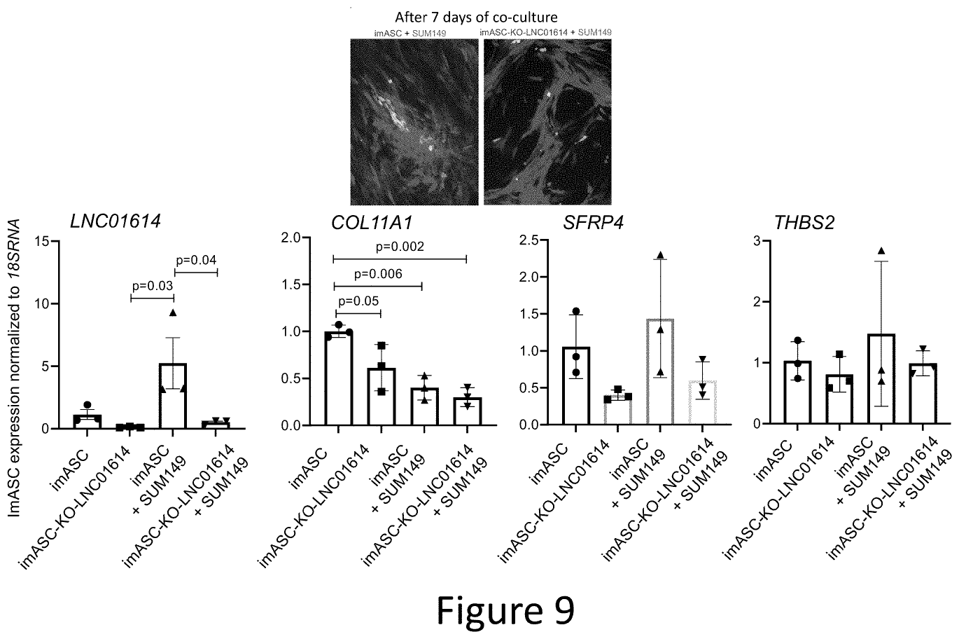

Changes in imASC induced by co-culture with SUM149 cancer cells, a comparatively less aggressive cell line in which the epithelial-mesenchymal transition (EMT) has not yet occurred, were measured (FIG. 9). After 7 days of co-culture, the morphology of imASC and LINC01614-KO imASC was not distinguishable. However, SUM149 looked different in the two co-cultures. It was apparent that SUM149 cultured with imASC have undergone the EMT based on their spindle shape. In contrast, SUM149 cultured with LINC01614-KO imASC were still round sand-made cobble-stone colonies, indicating that EMT had not occurred. This suggests that LINC01614 expression in ASC is required for their property to induce EMT in cancer cells. An induction of LINC01614, SFRP4 and THBS2 expression were observed in imASC co-cultured with SUM149 for 7 days. In contrast, these genes were not induced in LINC01614-KO imASC co-cultured with SUM149. SUM149 did not further induce COL11A1 expression in imASC; COL11A1 expression was the lowest in LINC01614-KO imASC co-cultured with SUM149 (FIG. 9).

### REFERENCES

1. Kim H, Watkinson J, Varadan V, Anastassiou D. Multi-cancer computational analysis reveals invasion-associated variant of desmoplastic reaction involving INHBA, THBS2 and COL11A1. BMC Medical Genomics. 2010 Nov. 3; 3(1):51.
2. Schuetz C S, Bonin M, Clare S E, Nieselt K, Sotlar K, Walter M, et al. Progression-specific genes identified by expression profiling of matched ductal carcinomas in situ and invasive breast tumors, combining laser capture microdissection and oligonucleotide microarray analysis. Cancer Res. 2006 May 15; 66 (10):5278-86.
3. Bignotti E, Tassi R A, Calza S, Ravaggi A, Bandiera E, Rossi E, et al. Gene expression profile of ovarian serous papillary carcinomas: identification of metastasis-associated genes. Am J Obstet Gynecol. 2007 March; 196(3):245.e1-11.
4. Badea L, Herlea V, Dima S O, Dumitrascu T, Popescu I. Combined gene expression analysis of whole-tissue and microdissected pancreatic ductal adenocarcinoma identifies genes specifically overex-pressed in tumor epithelia. Hepatogastroenterology. 2008 December; 55(88):2016-27.
5. Wang M, Zhao Y, Zhang B. Efficient Test and Visualization of Multi-Set Intersections.

Scientific Reports. 2015 Nov. 25; 5(1):16923.
6. Shen L, Yang M, Lin Q, Zhang Z, Zhu B, Miao C. COL11A1 is overexpressed in recurrent non-small cell lung cancer and promotes cell proliferation, migration, invasion and drug resistance. Oncology Reports. 2016 Aug. 1; 36(2):877-85.
7. Garci′a-Pravia C, Galván) J A, Gutierrez-Corral N, Solar-Garci′ a L, Garci′a-Perez E, Garci′a-Ocaña M, et al. Overexpression of COL11A1 by Cancer-Associated Fibroblasts: Clinical Relevance of a Stromal Marker in Pancreatic Cancer. PLOS ONE. 2013 Oct. 23; 8(10):e78327.
8. Wu Y-H, Chang T-H, Huang Y-F, Chen C-C, Chou C-Y. COL11A1 confers chemoresistance on ovarian cancer cells through the activation of Akt/c/EBPβ pathway and PDK1 stabilization. Oncotarget. 2015 Jun. 10; 6(27):23748-63.
9. Chen P-C, Tang C-H, Lin L-W, Tsai C-H, Chu C-Y, Lin T-H, et al. Thrombospondin-2 promotes prostate cancer bone metastasis by the up-regulation of matrix metalloproteinase-2 through down-regulating miR-376c expression. Journal of Hematology & Oncology. 2017 Jan. 25; 10(1):33.
10. Seder C W, Hartojo W, Lin L, Silvers A L, Wang Z, Thomas D G, et al. INHBA Overexpression Promotes Cell Proliferation and May Be Epigenetically Regulated in Esophageal Adenocarcinoma. Journal of Thoracic Oncology. 2009 Apr. 1; 4(4):455-62.
11. Wang Q, Wen Y-G, Li D-P, Xia J, Zhou C-Z, Yan D-W, et al. Upregulated INHBA expression is associated with poor survival in gastric cancer. Med Oncol. 2012 Marl; 29(1):77-83.
12. Tu H, Li J, Lin L, Wang L. COL11A1 Was Involved in Cell Proliferation, Apoptosis and Migration in Non-Small Cell Lung Cancer Cells. Journal of Investigative Surgery. 2020 Nov. 5; 0(0):1-6.
13. Wang X, Zhang L, Li H, Sun W, Zhang H, Lai M. THBS2 is a Potential Prognostic Biomarker in Colorectal Cancer. Scientific Reports. 2016 Sep. 16; 6(1):33366.
14. Li X, Yu W, Liang C, Xu Y, Zhang M, Ding X, et al. INHBA is a prognostic predictor for patients with colon adenocarcinoma. BMC Cancer. 2020 Apr. 15; 20(1):305.
15. Seder C W, Hartojo W, Lin L, Silvers A L, Wang Z, Thomas D G, et al. Upregulated INHBA Expression May Promote Cell Proliferation and Is Associated with Poor Survival in Lung Adenocarcinoma. Neopla-sia. 2009 April; 11(4):388-96.
16. Verhaak R G W, Tamayo P, Yang J-Y, Hubbard D, Zhang H, Creighton C J, et al. Prognostically relevant gene signatures of high-grade serous ovarian carcinoma. J Clin Invest. 2013 January; 123(1):517-25.
17. Moffitt R A, Marayati R, Flate E L, Volmar K E, Loeza S G H, Hoadley K A, et al. Virtual microdissection identifies distinct tumor- and stroma-specific subtypes of pancreatic ductal adenocarcinoma. Nature Genetics. 2015 October; 47(10):1168-78.
18. Saelens W, Cannoodt R, Todorov H, Saeys Y. A comparison of single-cell trajectory inference methods. Nature Biotechnology. 2019 May; 37(5):547-54.
19. Peng J, Sun B-F, Chen C-Y, Zhou J-Y, Chen Y-S, Chen H, et al. Single-cell RNA-seq highlights intra-tumoral heterogeneity and malignant progression in pancreatic ductal adenocarcinoma. Cell Research. 2019 September; 29(9):725-38.
20. Cheng W-Y, Yang T-H O, Anastassiou D. Biomolecular Events in Cancer Revealed by Attractor Meta-genes. PLOS Computational Biology. 2013 Feb. 21; 9(2):e1002920.
21. Cheng W-Y, Ou Yang T-H, Anastassiou D. Development of a prognostic model for breast cancer survival in an open challenge environment. Sci Transl Med. 2013 Apr. 17; 5(181):181ra50.
22. McCarthy N. Rising to the challenge. Nature Reviews Cancer. 2013 June; 13(6):378-378.
23. Boquest A C, Shandadfar A, Frønsdal K, Sigurjonsson O, Tunheim S H, Collas P, et al. Isolation and Transcription Profiling of Purified Uncultured Human Stromal Stem Cells: Alteration of Gene Expression after In Vitro Cell Culture. MBoC. 2005 Jan. 5; 16(3):1131-41.
24. Vijay J, Gauthier M-F, Biswell R L, Louiselle D A, Johnston J J, Cheung W A, et al. Single-cell analysis of human adipose tissue identifies depot- and disease-specific cell types. Nature Metabolism. 2020 January; 2 (1):97-109.
25. J D, Am S, J W, R F, N Z, A C, et al. Expression of secreted frizzled-related protein 4 (SFRP4) in primary serous ovarian tumours. Eur J Gynaecol Oncol. 2009 Jan. 1; 30(2):133-41.
26. Sandsmark E, Andersen M K, Bofin A M, Bertilsson H, Drablos F, Bathen T F, et al. SFRP4 gene expression is increased in aggressive prostate cancer. Scientific Reports. 2017 Oct. 27; 7(1):14276.
27. Ohnishi S, Okabe K, Obata H, Otani K, Ishikane S, Ogino H, et al. Involvement of tazarotene-induced gene 1 in proliferation and differentiation of human adipose tissue-derived mesenchymal stem cells. Cell Proliferation. 2009; 42(3):309-16.
28. Jing C, El-Ghany M A, Beesley C, Foster C S, Rudland P S, Smith P, et al. Tazarotene-Induced Gene 1 (TIG1) Expression in Prostate Carcinomas and Its Relationship to Tumorigenicity. JNCI: Journal of the National Cancer Institute. 2002 Apr. 3; 94(7):482-90.
29. Oldridge E E, Walker H F, Stower M J, Simms M S, Mann V M, Collins A T, et al. Retinoic acid represses invasion and stem cell phenotype by induction of the metastasis suppressors RARRES1 and LXN. Oncogenesis. 2013 April; 2(4):e45-e45.
30. Kashiwagi M, Friess H, Uhl W, Berberat P, Abou-Shady M, Martignoni M, et al. Group II and IV phos-pholipase A2 are produced in human pancreatic cancer cells and influence prognosis. Gut. 1999 October; 45(4):605-12.
31. Buhmeida A, Bendardaf R, Hilska M, Laine J, Collan Y, Laato M, et al. PLA2 (group IIA phospholipase A2) as a prognostic determinant in stage II colorectal carcinoma. Annals of Oncology. 2009 Jul. 1; 20 (7):1230-5.
32. Cai H, Chiorean E G, Chiorean M V, Rex D K, Robb B W, Hahn N M, et al. Elevated Phospholipase A2 Activities in Plasma Samples from Multiple Cancers. PLOS ONE. 2013 Feb. 22; 8(2):e57081.
33. Nallanthighal S, Rada M, Heiserman J P, Cha J, Sage J, Zhou B, et al. Inhibition of collagen XI alpha 1-induced fatty acid oxidation triggers apoptotic cell death in cisplatin-resistant ovarian cancer. Cell Death & Disease. 2020 Apr. 20; 11(4):1-12.
34. Koundouros N, Poulogiannis G. Reprogramming of fatty acid metabolism in cancer. British Journal of Cancer. 2020 January; 122(1):4-22.
35. Street K, Risso D, Fletcher R B, Das D, Ngai J, Yosef N, et al. Slingshot: cell lineage and pseudotime inference for single-cell transcriptomics. BMC Genomics [Internet]. 2018 Jun. 19 [cited 2020 Jun. 7]; 19. Available from:
36. Puram S V, Tirosh I, Parikh A S, Patel A P, Yizhak K, Gillespie S, et al. Single-Cell Transcriptomic Analysis of Primary and Metastatic Tumor Ecosystems in Head and Neck Cancer. Cell. 2017 Dec. 14; 171 (7):1611-1624.e24.
37. Shih A J, Menzin A, Whyte J, Lovecchio J, Liew A, Khalili H, et al. Identification of grade and origin specific cell populations in serous epithelial ovarian cancer by single cell RNA-seq. PLOS ONE. 2018 Nov. 1; 13(11):e0206785.
38. Lambrechts D, Wauters E, Boeckx B, Aibar S, Nittner D, Burton O, et al. Phenotype molding of stromal cells in the lung tumor microenvironment. Nature Medicine. 2018 August; 24(8):1277-89.
39. Karaayvaz M, Cristea S, Gillespie S M, Patel A P, Mylvaganam R, Luo C C, et al. Unravelling subclonal heterogeneity and aggressive disease states in TNBC through single-cell RNA-seq. Nature Communications. 2018 Sep. 4; 9(1):3588.
40. Vazquez-Villa F, Garci′a-Ocaña M, Galvan J A, Garci′a-Marti′nez J, Garci′a-Pravia C, Menéndez-Rodri′-guez P, et al. COL11A1/(pro)collagen 11A1 expression is a remarkable biomarker of human invasive carcinoma-associated stromal cells and carcinoma progression. Tumor Biol. 2015 Apr. 1; 36(4):2213-22.
41. Jia D, Liu Z, Deng N, Tan T Z, Huang R Y-J, Taylor-Harding B, et al. A COL11A1-correlated pan-cancer gene signature of activated fibroblasts for the prioritization of therapeutic targets. Cancer Letters. 2016 Nov. 28; 382(2):203-14.
42. Qian J, Olbrecht S, Boeckx B, Vos H, Laoui D, Etlioglu E, et al. A pan-cancer blueprint of the heterogeneous tumor microenvironment revealed by single-cell profiling. Cell Research. 2020 September; 30(9):745-62.
43. Yeung T-L, Leung C S, Yip K-P, Sheng J, Vien L, Bover L C, et al. Anticancer Immunotherapy by MFAP5 Blockade Inhibits Fibrosis and Enhances Chemosensitivity in Ovarian and Pancreatic Cancer. Clin Cancer Res. 2019 Nov. 1; 25(21):6417-28.
44. Ritchie M E, Phipson B, Wu D, Hu Y, Law C W, Shi W, et al. limma powers differential expression analyses for RNA-sequencing and microarray studies. Nucleic Acids Research. 2015 Apr. 20; 43(7):e47-e47.
45. Quail D F, Dannenberg A J. The obese adipose tissue microenvironment in cancer development and progression. Nature Reviews Endocrinology. 2019 March; 15(3):139-54.
46. Lengyel E, Makowski L, DiGiovanni J, Kolonin M G. Cancer as a Matter of Fat: The Crosstalk between Adipose Tissue and Tumors. Trends Cancer. 2018; 4(5):374-84.
47. Cozzo A J, Fuller A M, Makowski L. Contribution of Adipose Tissue to Development of Cancer. Compr Physiol. 2017 12; 8(1):237-82.
48. Sahai E, Astsaturov I, Cukierman E, DeNardo D G, Egeblad M, Evans R M, et al. A framework for advancing our understanding of cancer-associated fibroblasts. Nature Reviews Cancer. 2020 March; 20 (3):174-86.
49. Dominguez C X, Mu″ller S, Keerthivasan S, Koeppen H, Hung J, Gierke S, et al. Single-Cell RNA Sequencing Reveals Stromal Evolution into LRRC15+ Myofibroblasts as a Determinant of Patient Response to Cancer Immunotherapy. Cancer Discov. 2020 Feb. 1; 10(2):232-53.
50. Ohlund D, Handly-Santana A, Biffi G, Elyada E, Almeida A S, Ponz-Sarvise M, et al. Distinct populations of inflammatory fibroblasts and myofibroblasts in pancreatic cancer. J Exp Med. 2017 Mar. 6; 214 (3):579-96.
51. Elyada E, Bolisetty M, Laise P, Flynn W F, Courtois E T, Burkhart R A, et al. Cross-Species Single-Cell Analysis of Pancreatic Ductal Adenocarcinoma Reveals Antigen-Presenting Cancer-Associated Fibroblasts. Cancer Discov. 2019; 9(8):1102-23.
52. Chen Z, Zhou L, Liu L, Hou Y, Xiong M, Yang Y, et al. Single-cell RNA sequencing highlights the role of inflammatory cancer-associated fibroblasts in bladder urothelial carcinoma. Nature Communications. 2020 Oct. 8; 11(1):5077.
53. Helms E, Onate M K, Sherman M H. Fibroblast Heterogeneity in the Pancreatic Tumor Microenviron-ment. Cancer Discov. 2020 May 1; 10(5):648-56.
54. Wang Y, Liang Y, Xu H, Zhang X, Mao T, Cui J, et al. Single-cell analysis of pancreatic ductal adenocar-cinoma identifies a novel fibroblast subtype associated with poor prognosis but better immunotherapy response. Cell Discov. 2021 May 25; 7(1):1-17.
55. Stuart T, Butler A, Hoffman P, Hafemeister C, Papalexi E, Mauck W M, et al. Comprehensive Integration of Single-Cell Data. Cell. 2019 Jun. 13; 177(7):1888-1902.e21.
56. Kiselev V Y, Andrews T S, Hemberg M. Challenges in unsupervised clustering of single-cell RNA-seq data. Nature Reviews Genetics. 2019 May; 20(5):273-82.
57. Thomas M. Cover J A T. Elements Of Information Theory 2nd Ed [Internet]. 2006 [cited 2021 Apr. 6].
58. Daub C O, Steuer R, Selbig J, Kloska S. Estimating mutual information using B-spline functions—an improved similarity measure for analysing gene expression data. BMC Bioinformatics. 2004 Aug. 31; 5 (1):118.
59. Zhu K, Ou Yang T-H, Done V, Zheng T, Anastassiou D. Meta-analysis of expression and methylation signatures indicates a stress-related epigenetic mechanism in multiple neuropsychiatric disorders. Translational Psychiatry. 2019 Jan. 22; 9(1):1-12.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

Various publications and nucleic acid and amino acid sequence accession numbers are cited herein, the contents and full sequences of which are hereby incorporated by reference herein in their entireties.

We claim:

1. A method of treating cancer in a subject comprising: administering a therapeutically effective amount of a cancer transition inhibitor to the subject, wherein the cancer transition inhibitor reduces the expression level of a marker of the transition of adipose-derived stromal cells (ASCs) to COL11A1-expressing cancer-associated fibroblasts (CAFs).

2. The method of claim 1, wherein the cancer transition inhibitor is a polypeptide, a nucleic acid, a small molecule, or a combination of two or more thereof.

3. The method of claim 1, wherein the cancer transition inhibitor reduces the expression level of the marker by introducing an indel into the coding sequence of the marker.

4. The method of claim 3, wherein cancer transition inhibitor is a transposase/transposon, a Zinc Finger nuclease, a TALEN, or an RNA-guided nuclease.

5. The method of claim 1, wherein the marker is a long non-coding ribonucleic acid (lncRNA), a micro ribonucleic acid (miRNA), RARRES1, SFRP4, COL11A1, INHBA, THBS2 or a combination thereof.

6. The method of claim 5, wherein the lncRNA is LINC01614, AC134312.5, AC009093.1, LINC01615, or combinations thereof.

7. The method of claim 5, wherein the miRNA is hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof.

8. A pharmaceutical composition comprising: a therapeutically effective amount of a cancer transition inhibitor capable of reducing the expression level of a marker of a transition of ASCs to COL11A1-expressing CAFs and a pharmaceutically acceptable excipient.

9. The pharmaceutical composition of claim 8, wherein the cancer transition inhibitor is a polypeptide, a nucleic acid, a small molecule, or a combination of two or more thereof.

10. The pharmaceutical composition of claim 8, wherein the marker is an lncRNA, an miRNA, RARRES1, SFRP4, COL11A1, INHBA, THBS2 or a combination thereof.

11. The pharmaceutical composition of claim 10, wherein the lncRNA is LINC01614, AC134312.5, AC009093.1, LINC01615, or combinations thereof.

12. The pharmaceutical composition of claim 10, wherein the miRNA is hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof.

13. A method for determining the prognosis of a subject having cancer, comprising:
(a) obtaining a sample from the subject, and
(b) determining an expression level of a marker related to a transition of ASCs to COL11A1-expressing CAFs, wherein increased expression of the marker in the sample relative to a reference control is indicative of a poor prognosis.

14. The method of claim 13, wherein the marker is an lncRNA, an miRNA, RARRES1, SFRP4, COL11A1, INHBA, THBS2 or a combination thereof.

15. The method of claim 14, wherein the lncRNA is LINC01614, AC134312.5, AC009093.1, LINC01615, or combinations thereof.

16. The method of claim 14, wherein the miRNA is hsa-mir-199a-1, hsa-mir-199b, hsa-mir-199a-2, hsa-mir-493, hsa-mir-134, hsa-mir-382, hsa-mir-127, hsa-mir-708, hsa-mir-379, hsa-mir-409, hsa-mir-214, or combinations thereof.

17. The method of claim 13, wherein the sample comprises tissue obtained from blood, bladder, breast, colon, brain, kidney, liver, lung, esophagus, gall-bladder, ovary, pancreas, stomach, cervix, thyroid, prostate, or skin of the subject.

18. The method of claim 13, further comprises comparing the expression level of the marker of the sample with an expression level of a reference control, wherein the reference control is a healthy cell that has wild-type RARRES1, SFRP4, COL11A1, INHBA, and/or THBS2 expression.


SEQUENCE LISTING (11 sequences)

SEQ ID NO: 1 - DNA, 20 bp, Artificial Sequence
  Description of Artificial Sequence Synthetic primer
tcaaccaagagcgaagccaa

SEQ ID NO: 2 - DNA, 20 bp, Artificial Sequence
  Description of Artificial Sequence Synthetic primer
ttggacacagaccctagcac

SEQ ID NO: 3 - DNA, 23 bp, Artificial Sequence
  Description of Artificial Sequence Synthetic primer
tggtgatcagaatcagaagttcg

SEQ ID NO: 4 - DNA, 23 bp, Artificial Sequence
  Description of Artificial Sequence Synthetic primer
aggagagttgagaattgggaatc

SEQ ID NO: 5 - DNA, 22 bp, Artificial Sequence
  Description of Artificial Sequence Synthetic primer
cagtctgagcaagtgtgacacc

SEQ ID NO: 6 - DNA, 22 bp, Artificial Sequence
  Description of Artificial Sequence Synthetic primer
ttgcagagacggatgcgtgtga

SEQ ID NO: 7 - DNA, 22 bp, Artificial Sequence
  Description of Artificial Sequence Synthetic primer
ctatgaccgtggcgtgtgcatt

SEQ ID NO: 8 - DNA, 24 bp, Artificial Sequence
  Description of Artificial Sequence Synthetic primer
gcttaggcgtttacagtcaacatc

SEQ ID NO: 9 - DNA, 22 bp, Artificial Sequence
  Description of Artificial Sequence Synthetic primer
aagtccctgccctttgtacaca

SEQ ID NO: 10 - DNA, 21 bp, Artificial Sequence
  Description of Artificial Sequence Synthetic primer
gatccgagggcctcactaaac

SEQ ID NO: 11 - DNA, 25 bp, Artificial Sequence
  Description of Artificial Sequence Synthetic oligonucleotide
caccggtgtaaggtactcaagtgct